(19) United States
(12) Patent Application Publication
(10) Pub. No.: US 2021/0142867 A1
(43) Pub. Date: May 13, 2021

(54) Accurate And Robust Information-Deconvolution From Bulk Tissue Transcriptomes

(71) Applicant: Regeneron Pharmaceuticals, Inc., Tarrytown, NY (US)

(72) Inventors: Tao Yang, Tarrytown, NY (US); Yu Bai, Tarrytown, NY (US); Wen Fury, Tarrytown, NY (US); Gurinder Atwal, Tarrytown, NY (US)

(21) Appl. No.: 17/091,484

(22) Filed: Nov. 6, 2020

Related U.S. Application Data
(60) Provisional application No. 62/932,593, filed on Nov. 8, 2019.

(51) Int. Cl. G16B 30/00; G16B 5/00

(52) U.S. Cl. CPC G16B 30/00; G16B 5/00

(57) ABSTRACT

This disclosure relates to methods for deconvolving bulk or spatial RNA-sequencing data, computer readable medium storing processor-executable instructions adapted to deconvolve bulk or spatial RNA-sequencing data, and systems for deconvolving bulk or spatial RNA-sequencing data to characterize cell type compositions.

### FIELD

This disclosure relates generally to sequencing technology. More specifically, this disclosure relates to methods for deconvolving the sequencing data of a mixture sample and to related apparatuses and related computer readable medium storing processor-executable instructions to provide the mixing composition characteristics.

### BACKGROUND

In molecular biology, messenger RNA (mRNA) is a large family of RNA molecules that convey genetic information from deoxyribonucleic acid (DNA) to the ribosome, where they specify the amino acid sequence of the protein products of gene expression. The RNA polymerase enzyme transcribes genes into primary transcript mRNA (known as pre-mRNA) leading to processed, mature mRNA. This mature mRNA is then translated into a polymer of amino acids: a protein, as summarized in the central dogma of molecular biology.

As in DNA, mRNA genetic information is encoded in the sequence of nucleotides, which are arranged into codons consisting of three base pairs each. RNA sequencing (RNA-seq) is the process of determining the sequence of nucleotides in a strand of RNA. Each codon encodes a specific amino acid, except the stop codons, which terminate protein synthesis. This process of translation of codons into amino acids requires two other types of RNA: transfer RNA (tRNA), which mediates recognition of the codon and provides the corresponding amino acid, and ribosomal RNA (rRNA), which is the central component of the ribosome's protein-manufacturing machinery.

Bulk tissue RNA-seq is a widely adopted method applied to understand genome-wide transcriptomic variations in different states, such as normal or disease conditions. Since bulk tissues often consist of different cell types, bulk RNA-seq measures the average expression of each gene, which is the sum of cell-type specific gene expression weighted by cell-type proportions. Knowledge of cell-type compositions and their proportions in intact tissues is important to understand the biology of the tissue. It can lead to the characterization of tissue states in terms of cellular composition difference, and adjusting for these variations can direct better downstream analysis. However, the bulk tissue RNA-seq data do not directly provide cell type composition information. This is because the gene expression levels of each mixing cell type are not elucidated in the bulk data.

Recent breakthroughs in spatial transcriptomics methods enable characterizing whole transcriptome-wise gene expressions at spatially resolved locations in a tissue section. However, it remains challenging to reach a single cell resolution while measuring tens of thousands of genes transcriptome-wise. Some widely used technologies can achieve a resolution of 50-100 μm, equivalent to 3-30 cells depending on the tissue type. The transcripts therein may originate from one or more cell types. Unlike the bulk RNA-seq, the profiling data at each spot contains substantial dropouts as merely a few cells are sequenced, imposing additional challenges to demystify the cell type content. Both the bulk RNA-seq and spatial transcriptomics data at the multi-cell resolution are compound RNA-seq data wherein the mixing proportions of the cell types are unknown.

The rapid development of single-cell RNA-seq (scRNA-seq) technologies have allowed for cell-type specific transcriptome profiling. Although cell-type compositions and proportions can be directly obtained from scRNA-seq data and, thus, such technologies can provide the information missing from the compound RNA-seq data, the technologies have low sensitivity and unacceptably large noise due to the high dropout rate and the cell-to-cell variability. Consequently, scRNA-seq technologies require a large number of cells (thousands to tens of thousands) to ensure statistical significance in the results. In addition, the cells must remain viable during capture. These requirements render the scRNA-seq technologies costly, prohibiting their application in clinical studies that involve a large number of subjects or cannot allow real time tissue disassociation and cell capture. Furthermore, scRNA-seq technologies are not well suited to characterizing cell-type proportions in solid tissues because the cell dissociation and capture steps can be biased towards certain cell types.

Sequencing at the single cell level is not always feasible, and it has its own limitations as described. Besides, there are also many existing bulk RNA-seq data that can benefit from the information obtained from cell type composition. Thus, computational approaches have been developed to deconvolve cell type proportions from the bulk tissue RNA-seq data. The deconvolution process is essentially an optimization problem, with the mixing proportion of a finite number of cell types being the parameters to optimize. A goal is to minimize the difference between the observed gene expression in the bulk tissue RNA-seq data with their corresponding expected values that are computed as the sum of the pre-defined cell type specific expression weighted by the mixing proportion parameters. The best mixing proportion that minimizes the difference is the final output.

One such computational method is disclosed in Wang et al., “Bulk tissue cell type deconvolution with multi-subject single-cell expression reference,” Nature Communications (Published Online Jan. 22, 2019). The authors introduce a “MUlti-Subject Single Cell deconvolution” (MuSiC) method (code available) that uses cross-subject scRNA-seq to estimate cell-type proportions in bulk RNA-seq data. More specifically, MuSiC is a weighted non-negative least squares regression (W-NNLS), which does not require pre-selected marker genes. MuSiC uses cross-subject variation that reflects the gene stability to weight the genes. The iterative estimation procedure automatically imposes more weight on stable genes and less weight on variable genes. Because it is a linear regression-based method, genes showing large cross-subject variations will have low leverage, thus having less influence on the regression, whereas the most influential genes are those with high stability weight. MuSiC is one of many alternative computational methods that are available.

In addition, most methods limit the data to a pre-defined set of cell-type specific genes and their outputs vary depending on different choices of such gene sets, rendering the results less objective and less robust. For example, CIBERSORT, although well-known, has been reported to have poor sensitivity (see, world wide web at “nature.com/articles/s41467-018-08023-x”). Furthermore, most existing methods are only suitable for relatively simple applications such as peripheral blood mononuclear cells (PBMC) and the pancreas, in which only a handful number of cell types need to be considered, or the difference between cell types is rather large. Their performance in complex tissues with tens of different cell types or cell subtypes of subtle difference is questionable.

In view of the foregoing, there is a need for improved methods of accurate and robust deconvolution from bulk tissue transcriptomes and spatial transcriptomes at multi-cell resolution.

### SUMMARY

The present disclosure provides methods (including computer-implemented methods), computer programs, computer systems, and apparatus for deconvolving bulk RNA-sequencing data. A goal is to meet the need for obtaining accurate and robust cell-type proportion estimations from bulk tissue transcriptomes.

The present disclosure provides methods for deconvolving bulk RNA-sequencing data using pre-defined cell type specific expression obtained from single cell RNA-seq of cell types that are relevant to the bulk tissues. The methods comprise any one or more of: i) from single cell RNA-seq data, selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) from single cell RNA-seq data, computing cell type-specific weights for each selected gene within the subset of most variably expressed genes within the normalized matrix of counts-based sequencing data and using cell type annotation; iii) from single cell RNA-seq data, fitting a cross-sample distribution for each of the most variably expressed genes for each cell type from the counts-based sequencing data matrix, the subset of the most variably expressed genes, and the cell type annotation, and defining a mixed single-cell distribution with proportion parameters; iv) fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; v) defining a loss function between the bulk distribution and the mixed single-cell distribution; and vi) applying the loss function to estimate cell type proportions in the bulk RNA-sequencing data; thereby inferring the percentage of cell types in the bulk RNA-sequencing data. In some embodiments, the counts-based sequencing data is single-cell RNA-sequencing data, the counts-based sequencing counts is single-cell RNA-sequencing counts, and the counts-based sequencing data matrix is single-cell RNA-sequencing data matrix. In some embodiments, the counts-based sequencing data is ATAC-seq data, the counts-based sequencing counts is ATAC-seq counts, and the counts-based sequencing data matrix is ATAC-seq data matrix. In some embodiments, the cross-sample distribution for each cell type and for each of the subset of the most variably expressed genes from the counts-based sequencing data matrix is a cross-sample Gaussian distribution. In some embodiments, the bulk distribution for each subset of the most variably expressed genes from a normalized bulk matrix and a subset of the most variably expressed genes is a bulk Gaussian distribution.

The present disclosure also provides methods for deconvolving bulk RNA-sequencing data comprise any one or more of six exemplary steps: i) obtaining input from three sources (bulk or spatial RNA-seq data, single cell RNA-seq data, and cell type annotations) and selecting a subset of the most variably expressed genes from a matrix of counts-based sequencing data, wherein the matrix of counts-based sequencing data comprises counts-based sequencing counts against each gene within a plurality of genes for a fixed number of cells; ii) estimating the mean and dispersion parameters of the expression per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities and using the model to estimate cell type proportions in the bulk or spatial RNA-sequencing data; thereby inferring the percentage of cell types in the bulk RNA-sequencing data.

The present disclosure also provides computer readable medium storing processor-executable instructions adapted to cause one or more computing devices to deconvolve bulk RNA-sequencing data by any of the methods described herein.

The present disclosure also provides systems comprising one or more processors and a memory having processor executable instructions that, when executed by the one or more processors, cause the apparatus to deconvolve bulk RNA-sequencing data by any of the methods described herein.

It is to be understood that both the foregoing general description and the following detailed description are exemplary, but are not restrictive, of the disclosure.

### BRIEF DESCRIPTION OF THE DRAWINGS

The disclosure is best understood from the following description when read in connection with the accompanying drawings. The patent or application file contains at least one figure of the drawings executed in color. Copies of this patent or patent application publication with color figures will be provided by the U.S. Patent and Trademark Office upon request and payment of the necessary fee. Included in the drawings are the following figures:

FIG. 1 shows an overview of the workflow of one embodiment of the disclosed methods.

FIGS. 2A, 2B, and 2C illustrate three different hypothetical gene expressions versus cell type patterns as a basis for selecting the most informative genes in one embodiment of the disclosed methods.

FIG. 3 illustrates computation of the variance of genes across all cell types and selection of the top 2,500 variable genes in one embodiment of the disclosed methods.

FIG. 4 illustrates computation of the overall or total mean variance and the within cell-type mean variance in one embodiment of the disclosed methods.

FIG. 5 shows estimation of the cell-type specific variances and means by fitting a Gaussian distribution in one embodiment of the disclosed methods.

FIG. 6 shows estimation of the bulk data cross-sample variances and means by fitting a Gaussian distribution in one embodiment of the disclosed methods.

FIG. 7 illustrates a comparison between the mixture distribution of single-cell data and the distribution of the bulk-cell data in one embodiment of the disclosed methods.

FIG. 8 shows results of Gaussian distribution fits following the application of step 3 of the embodiment of the disclosed method shown in FIG. 1 to an illustrative example.

FIG. 9 shows results of Gaussian distribution fits following the application of step 4 of the embodiment of the disclosed method shown in FIG. 1 to the illustrative example.

FIG. 10 shows results of applying step 5 in one embodiment of the disclosed method shown in FIG. 1 to the illustrative example, namely, defining a model using the proportion parameters, weights, and distributions of each gene learned from the single-cell and from the bulk-cell data of the example.

FIG. 11 shows an overview of the workflow of another embodiment of the disclosed methods (AdRoit methods).

FIG. 12A illustrates two options for selecting the most informative genes during the first step of the methods disclosed in FIG. 11.

FIG. 12B provides a hypothetical example illustrating the type of cells that will be selected using the methods disclosed in FIG. 11.

FIG. 13 illustrates the second step of the methods disclosed in FIG. 11 of estimating the mean and dispersion parameters by fitting a negative binomial distribution for each gene in a cell type.

FIG. 14 provides a hypothetical example demonstrating the effect of the gene-wise scaling factor applied during the fifth step of the methods disclosed in FIG. 11.

FIG. 15A is a summary of the human pancreatic islets cell compositions of 18 subjects, and FIG. 15B is a t-SNE graph showing the four cell types are distinct from each other.

FIGS. 16A, 16B, and 16C present graphs reflecting a comparison of accuracy of estimates to the true percentages among the estimates of the AdRoit method (FIG. 16A), the MuSiC method (FIG. 16B), and the NNLS method (FIG. 16C) for all cell types from the 18 subjects.

FIG. 17 is a table listing four, separate, statistical measurements (mAD, RMSD, and Spearman and Pearson correlations) calculated for each of the three graphs of FIGS. 16A, 16B, and 16C.

FIG. 18A is a summary of the human trabecular meshwork cell compositions of eight donors, and FIG. 18B is a t-SNE graph showing the distinction as well as similarity between cell types. The data was used to evaluate the disclosed methods against other conventional methods.

FIGS. 19A, 19B, and 19C present graphs reflecting a comparison of accuracy of estimates to the true percentages among the results of the AdRoit method (FIG. 19A), the MuSiC method (FIG. 19B), and the NNLS method (FIG. 19C) for the eight donors.

FIG. 20 is a table listing four, separate, statistical measurements (mAD, RMSD, and Spearman and Pearson correlations) calculated for each of the three graphs of FIGS. 19A, 19B, and 19C.

FIG. 21 shows a comparison of how much deviation the estimates are from the truth among the three methods. One dot represents a donor and one row is a cell type in the human trabecular meshwork.

FIG. 22 reflects estimated and true data calculated using both the AdRoit method and the MuSiC method for the human trabecular meshwork cell types.

FIG. 23 is a receiver operating characteristic (ROC) curve showing that the AdRoit method had a significantly higher area under curve (AUC) than the MuSiC method for detecting the human trabecular meshwork cell types, implying a higher sensitivity of AdRoit.

FIG. 24A is a summary of the cell composition of five mice, and FIG. 24B is a t-SNE graph of the cell types discovered in the mouse dorsal root ganglion single-cell data used. This data was later used to evaluate the disclosed methods against other conventional methods.

FIGS. 25A, 25B, and 25C present graphs reflecting a comparison of accuracy of estimates to the true cell percentages among the results of the AdRoit method (FIG. 25A), the MuSiC method (FIG. 25B), and the NNLS method (FIG. 25C) for the five mice.

FIG. 26 is a graphical presentation comparing the results of the AdRoit method, the MuSiC method, and the NNLS method on the mouse data using the mAD, RMSD, and Pearson and Spearman correlations as statistical measurements.

FIG. 27 is a graph showing that estimations based on the AdRoit method of cell-type percentages on real human islets bulk RNA-seq data are highly reproducible for repeated samples from the same donor.

FIG. 28 shows that cell type percentages of human islets data estimated using the Adroit method agree with the RNA-FISH measurements of cell-type percentages.

FIG. 29 shows that Beta cell proportion estimated using the Adroit method have a significant negative linear relationship with donors' HbA1C levels (including both healthy and T2D cells).

FIG. 30 shows that Beta cell proportion estimated using the Adroit method in T2D patients are significantly lower than in healthy subjects.

FIG. 31 compares estimations achieved by stereoscope and the AdRoit method on simulated spatial spots that contain five different PEP cell subtypes.

FIG. 32 compares the performance when the percent of cells is low using simulated data. A series of low percent PEP cells were simulated and mixing with other two PEP cell types. The result shows that the medians of the estimates achieved using the AdRoit method were close to the true proportions and closer than the estimates achieved using stereoscope.

FIG. 33 compares the detection rates of AdRoit method and stereoscope method using simulated spatial spots. The simulation include 6 different mixing schemes of cell types, each type of mixing contains a series of low percent cell type. The evaluation is to see how much of the low percent cell type was detected at each given low percent.

FIG. 34 illustrates the cell type content estimated by AdRoit method at each spatial spot of the mouse brain coronal tissue section.

FIG. 35 provides the ISH images of the Wfs1, Prox2, and Rarres2 genes from the Allen mouse brain atlas that validates the cell type locations showed in FIG. 34 are accurate.

### DESCRIPTION OF EMBODIMENTS

Various terms relating to aspects of the present disclosure are used throughout the specification and claims. Such terms are to be given their ordinary meaning in the art, unless otherwise indicated. Other specifically defined terms are to be construed in a manner consistent with the definitions provided in this document.

Unless otherwise expressly stated, it is in no way intended that any method or aspect set forth be construed as requiring that its steps be performed in a specific order. Accordingly, where a method claim does not specifically state in the claim or description that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

RNA sequencing technology may provide an unprecedented opportunity in learning disease mechanisms and discovering new treatment targets. Recent spatial transcriptomics methods further enable the transcriptome profiling at spatially resolved spots in a tissue section. In controlled experiments, it is often of great importance to know the variability of cell composition under treatment interventions. Understanding the cell type content in each tissue spot is also crucial to the spatial transcriptome data interpretation. Though single cell RNA-seq has the power to reveal cell type composition and expression heterogeneity in different cells, it remains costly and sometimes infeasible when live cells cannot be obtained or sufficiently dissociated. To leverage the bulk and spatial RNA-seq data when sequencing at the single-cell level is not feasible, presented herein are methods to estimate the proportions of each cell type in the bulk or spatial RNA-seq data using known single-cell seq data of relevant cell types, such as data available in the public domain. The methods described herein jointly models the gene-wise technology bias, genes' cell type specificity and cross-sample variability, thus, is more accurate and robust. The systematic benchmarking evaluation shows superior sensitivity and specificity to other existing methods, even in neuronal cells where there exist many closely related subtypes.

The methods disclosed herein provide a statistical way to estimate proportions of each cell type in bulk RNA-seq data using independently acquired expression profile of relevant cell types (often publicly available) obtained from counts-based sequencing technology, such as single-cell data. The methods are especially well-suited for detecting rare (proportions less than about 5%) cell types. One assumption in implementing the methods described herein is that the tissues used for the single-cell RNA-seq contain the same or no less cell types as what are in the bulk or spatial sequencing samples.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

The disclosed methods, apparatus, and computer readable medium aim to accurately and robustly estimate the proportion of cell types from bulk tissue transcriptomes. Existing counts-based sequencing data, such as single-cell RNA-sequencing data, are used as a reference with cell identity annotated. The unique distribution characteristics of highly informative genes are ascertained for each cell type, and the composition of the cell types is estimated from bulk tissue or spatial RNA-sequencing data without relying on marker selection. Important to the success of the disclosed methods, apparatus, and computer readable medium are that: 1) when the mixing proportion is estimated, the whole distribution of the gene expression value, or the mean and dispersion parameters that define the distribution, is considered, not only the means; 2) high weights are placed on genes that are more distinguishable across cell types, that is, genes with an expression highly specific to certain cell types; 3) low weights are placed on genes that are highly variable cross multiple samples; 4) an adaptively learning approach is used to estimate gene-wise scaling factors to address the platform difference between the bulk or spatial RNA-sequencing data and the single cell RNA-sequencing data and 5) a regularization term is included in the model to minimize the impact of the statistical collinearity. Among other features of the disclosed methods, apparatus, and computer readable medium, these five features combine for improvement over existing methods.

The present disclosure provides methods for deconvolving bulk RNA-sequencing data. In some embodiments, the methods comprise any one or more of the following six exemplary steps: i) selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) computing cell type-specific weights for each selected gene within the subset of most variably expressed genes within the normalized matrix of counts-based sequencing data and using cell type annotation; iii) fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; iv) fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; v) defining a loss function between the bulk distribution and the mixed single-cell distribution; and vi) applying the loss function to estimate cell type proportions in the bulk RNA-sequencing data; thereby inferring the percentage of cell types in the bulk RNA-sequencing data. These steps are illustrated in FIG. 1 (showing single-cell RNA-sequencing as the counts-based sequencing), which shows an overview of the workflow of one embodiment of the disclosed methods. Each of the steps is discussed below, in turn, with reference to the input, output, and purpose or rationale for each step. Each of these process steps can be carried out by a computing device, such as a computer. In some embodiments, all of the process steps are carried out by a computer.

In some embodiments, the methods comprise the first step. In some embodiments, the methods comprise the first step and one or more of the second, third, fourth, fifth, and sixth steps, or any combination of these additional steps. In some embodiments, the methods comprise the second step. In some embodiments, the methods comprise the second step and one or more of the first, third, fourth, fifth, and sixth steps, or any combination of these additional steps. In some embodiments, the methods comprise the third step. In some embodiments, the methods comprise the third step and one or more of the first, second, fourth, fifth, and sixth steps, or any combination of these additional steps. In some embodiments, the methods comprise the fourth step. In some embodiments, the methods comprise the fourth step and one or more of the first, second, third, fifth, and sixth steps, or any combination of these additional steps. In some embodiments, the methods comprise the fifth step. In some embodiments, the methods comprise the fifth step and one or more of the first, second, third, fourth, and sixth steps, or any combination of these additional steps. In some embodiments, the methods comprise the sixth step. In some embodiments, the methods comprise the sixth step and one or more of the first, second, third, fourth, and fifth steps, or any combination of these additional steps.

In some embodiments, the counts-based sequencing data is single-cell RNA-sequencing data, the counts-based sequencing counts is single-cell RNA-sequencing counts, and the counts-based sequencing data matrix is single-cell RNA-sequencing data matrix. In some embodiments, the counts-based sequencing data is ATAC-seq data, the counts-based sequencing counts is ATAC-seq counts, and the counts-based sequencing data matrix is ATAC-seq data matrix. In some embodiments, the cross-sample distribution for each cell type and for each of the subset of the most variably expressed genes from the counts-based sequencing data matrix is a cross-sample Gaussian distribution. In some embodiments, the bulk distribution for each subset of the most variably expressed genes from a normalized bulk matrix and a subset of the most variably expressed genes is a bulk Gaussian distribution. The methods described herein result in an inference of single-cell distribution proportions to the bulk RNA-sequencing data.

In some embodiments, the methods further comprise creating the matrix of counts-based sequencing counts against each gene within the plurality of genes for a fixed number of cells and normalizing the matrix. In some embodiments, the methods further comprise creating the bulk matrix of bulk RNA-sequencing counts and normalizing the bulk matrix. In some embodiments, the methods further comprise creating the matrix of counts-based sequencing counts against each gene within the plurality of genes for a fixed number of cells and normalizing the matrix, and creating the bulk matrix of bulk RNA-sequencing counts and normalizing the bulk matrix. In some embodiments, the methods further comprise obtaining cell type annotation. In some embodiments, the counts-based sequencing data is single-cell RNA-sequencing data, the counts-based sequencing counts is single-cell RNA-sequencing counts, and the counts-based sequencing data matrix is single-cell RNA-sequencing data matrix. In some embodiments, the counts-based sequencing data is ATAC-seq data, the counts-based sequencing counts is ATAC-seq counts, and the counts-based sequencing data matrix is ATAC-seq data matrix.

In some embodiments, the methods further comprise identifying the proportion of RNA from each cell type from which the bulk RNA-sequencing data were obtained. In some embodiments, the methods further comprise identifying the proportion of each cell type from which the bulk RNA-sequencing data were obtained. In some embodiments, the methods further comprise identifying the proportion of RNA from each cell type from which the bulk RNA-sequencing data were obtained and identifying the proportion of each cell type from which the bulk RNA-sequencing data were obtained.

### Step 1: Select Top “N” Highly Variable Genes

A purpose of the first step in the illustrative embodiment of the disclosed methods (FIG. 1) is to select the most informative genes. This step is applied to single-cell RNA-seq (scRNA-seq) data, but can be applied to any counts-based sequencing data as set forth herein. In scRNA-seq data, not all the genes in the genome will be informative about the identity of the particular cell. One reason is because many genes are essential to cell survival and other basic cell functions. These genes are universally expressed across many different cell types and not biologically differentiable by cell types. Further, due to technology limitations, scRNA-seq usually captures a few hundred to a couple of thousand genes from each cell, depending on the platform (10× or C1). The majority of the genes will have no readout across all cells. The genes in both cases will not contribute to the cell identity specification and, therefore, are not informative in deconvolving the bulk data.

FIGS. 2A, 2B, and 2C illustrate one reason why it is important to select the most informative genes, using graphs of expression (ordinate) versus cell type C₁, C₂, C₃, C₄, and C₅ (abscissa) for each of three hypothetical genes. FIG. 2A depicts an informative Gene 1 because the data within each cell type are relatively consistent and the data are distinguishable among the five cell types. The data of FIG. 2B are not helpful because the data within each cell type are too variable. The data of FIG. 2C are not helpful because the cell type-to-cell type data are not sufficiently different.

Known analysis methodologies can be used to choose the highly variable genes. See, e.g., A. Butler et al., “Integrating single-cell transcriptomic data across different conditions, technologies, and species,” Nat. Biotechnol. (2018) (A. Butler, Nat. Biotechnol.); and F. Wolf et al., “SCANPY: Large-scale single-cell gene expression data analysis,” Genome Biol. (2018). Normally, when single cells are computationally clustered based on scRNA-seq data, the top 2,000 highly variables genes will yield good separation between different cell types. It is recommended that somewhat more than this number of 2,000 genes be selected, however, because data processing can induce information loss. On the other hand, a balance should be maintained because selecting too many genes would introduce noise. Thus, in some embodiments, the top 2,500 highly variable genes are selected. More or less genes than that number can be selected depending upon the application (e.g., the cell type). The preferred number of variable genes to be selected can be predetermined by trial and error based on which number achieves the best validation. By “predetermined” is meant determined beforehand, so that the predetermined characteristic must be determined, i.e., chosen or at least known, in advance of some event. Preferably, the minimum and maximum number of highly variable genes that are selected range from about 1,000 to about 5,000. The genes are selected from the whole transcriptome measurable by RNA-seq technology. In the human transcriptome, there are about 25,000 genes; in the mouse transcriptome, about 20,000 genes.

Due to the well-known dispersion effect in RNA-seq data, directly computing the variation from the counts matrix would likely overestimate variance. The methods described herein address such overestimation by computing the variances from a variance stabilization transformed (VST) data matrix and select the genes based on a rank of these variances. FIG. 3 illustrates a representative computation of the variance of genes across all cell types and selection of the top 2,500 highly variable genes. The algorithm of this procedure is readily programed in the “Seurat” R package disclosed in A. Butler, Nat. Biotechnol. The function “Find VariableFeatures”, for example, is used to select the top 2,500 highly variable genes. Of course, other algorithms can be used for selection of the top 2,500 highly variable genes.

In this first step, as illustrated in FIG. 3, the single-cell expression matrix constitutes the input where rows represent genes and columns represent individual cell types. It is recommended, but not required, that the unique molecular identifiers (UMI) counts matrix (data from 10× platform) or RPKM (data from C1 platform) be used. The annotation of the cell types is not needed at this step. After VST is applied to the single-cell counts matrix, the standard deviation (represented by the Greek symbol sigma or “a”) is computed for each row (gene) to yield the 2,500 most highly variable genes. The standard deviation is a measure of the extent of deviation for a group of data as a whole. The standard deviation is calculated as follows: 1) calculate the mean or average; 2) for each number, subtract the mean and square the result; 3) calculate the mean of the squared differences (the variance); and 4) calculate the square root of that mean. The output from the first step of the disclosed method is the top “N” number (i.e., 2,500) of highly variable genes. The disclosed methods later confine computations to these N genes.

### Step 2: Compute Cell Type Specific Weights

The input to the second step in the illustrative embodiment of the disclosed methods is the same single-cell counts matrix as the first step, but can be any counts-based sequencing data matrix as set forth herein. The second step also requires as input, however, the cell identity information (i.e., cell type annotation) because the cell-type specific variance will be computed. One purpose of the second step is to quantify the importance of a gene on defining a cell type.

FIG. 4 illustrates a representational computation of the overall or total mean variance and the within cell-type mean variance. The mean variance is computed across cells within each cell type and compared to the total mean variance. For the same dispersion reason, the variance on log counts (1 added to zero counts) is computed. If a gene is stably expressed (i.e., meaning low mean variance) within a cell type, but varies substantially across all cells (i.e., meaning high mean variance), then that gene should be a good specifier for the cell type and, therefore, should receive a large weight.

To formally define the weight (“W”) of each cell type, let “N” be the total number of cells and “nₖ” be the number of cells in cell type “k.” Thus, N=n₁+n₂+n₃+ . . . . For a particular gene “i,” let σᵢ² be the variance across all cells, and σᵢₖ² be the variance of that gene within cell type k. Thus, the weight for a particular gene and cell type is expressed as:

$${w}_{ik} = \frac{{σ}_{i}^{2} / N - 1}{{σ}_{ik}^{2} / {n}_{k} - 1}$$

The numerator of the equation is the total mean variance; the denominator of the equation is the within cell-type mean variance. The weights are computed for all informative genes and all cell types, resulting in an I×K matrix, where I and K are the number of genes and number of clusters, respectively. The output from the second step of the disclosed method is a weight matrix in which the entries are cell-type specific weights for each gene. Rows of the matrix are genes and columns are cell types.

### Step 3: Fit Cell-Type Specific Gaussian Distributions Across Subjects

The input to the third step in the illustrative embodiment of the disclosed methods includes the single cell counts matrix and the list of highly variable genes from Step 1 as well as cell type annotation, but can be any counts-based sequencing data matrix as set forth herein. For multi-sample single cell data, the sample information should also be input.

Statistical tests analyze a particular set of data to make more general conclusions. There are several approaches to doing this, but the most common is based on assuming that data in the population have a certain continuous probability distribution. The distribution used most commonly is the bell-shaped Gaussian distribution, also called the normal distribution. Normal distributions are often used in the natural and social sciences to represent real-valued random variables whose distributions are not known. A random variable with a Gaussian distribution is said to be normally distributed and is called a normal deviate.

One of the features of the disclosed methods is that the methods use the whole distribution when estimating the mixing proportion. The distribution is obtained by fitting the normalized count to a distribution, such as a Gaussian distribution, and estimating the variance and mean for each gene. The process of “normalizing” involves adjusting values measured on different scales to a common scale (i.e., eliminating units of measurement), usually before averaging. FIG. 5 shows how the cell-type specific variances and means are estimated by fitting a Gaussian distribution.

The disclosed methods can use at least two ways to estimate the distribution, such as a Gaussian distribution, depending on whether multiple samples are available or not. If multiple samples are available, the cells are pooled by adding the read counts within cell type, forming a mega-cell for each cell type. Mega cells alleviate data sparsity and sampling variation due to technology limitations and, therefore, better represent the unique transcriptome profile of each specific cell type. Unfortunately, however, multiple samples are not always available. If multiple samples are unavailable, the disclosed methods estimate the variance by randomly portioning cells into multiple subgroups, pooling cells within each subgroup, and using them such that they are from different samples.

The disclosed methods next normalize the mega cell count matrix for each sample. The methods fundamentally follow the standard way to normalize RNA-seq data, as disclosed in A. Butler, Nat. Biotechnol., and in M. Love et al., “Moderated estimation of fold change and dispersion for RNA-seq data with DESeq2,” Genome Biol. (2014), in which the data are rescaled such that each mega cell has the same total number of reads, then the rescaled data are log transformed. The disclosed methods make some relatively small adjustments to the standard way, however, to achieve better results. First, log transformation ensures the approximation to a distribution, such as a Gaussian distribution, but greatly reduces the variances upon which the disclosed methods rely. To alleviate the downside, the total number of reads are rescaled to a large number (e.g., 10⁷). Second, typically 1 is added to the zeros in the matrix to avoid log errors, but at the same time it may unfavorably change the distribution especially for the lowly expressed genes. Although adding 1 yields acceptable results, adding a smaller number (e.g., 0.1) helps improve the estimation accuracy, and also speeds up the convergence of the algorithm to be described (below) in Step 5 of the method.

The output from the third step of the disclosed methods includes normalized expression matrices for mega cells and estimated mean and variance for each selected gene. In the example illustrated in the Figures, the output comprises five sets of 2,500 Gaussian curves (one for each cell type). At this point, the disclosed methods have completed the processing of the single-cell data and now turn to the bulk data.

### Step 4: Fit Gaussian Distribution to Bulk Data

The list of genes selected from Step 1 and the multi-sample bulk RNA-seq count matrix combine to form the input to the fourth step in the illustrative embodiment of the disclosed methods. Step 4 is very similar to Step 3. First, the total number of reads are rescaled to the same number as in the single cell analysis (e.g., 10⁷), then a small number (e.g., 0.1) is added to the zero counts and the log transformation is performed. Theoretically, rescaling the total reads for bulk RNA-seq data is unnecessary and will achieve similar results to unscaled data. In some embodiments, the disclosed methods include rescaling for the practical reason that a close number to the single cell total speeds up the algorithm convergence. FIG. 6 shows how the bulk data cross-sample variances and means are estimated by fitting a Gaussian distribution. The output from the fourth step of the disclosed methods includes a normalized expression matrix and estimated means and variances across samples for each selected gene.

### Step 5: Defining the Loss Function

The fifth step in the illustrative embodiment of the disclosed methods takes as input all the outputs from the previous steps, namely, the top highly variable genes, the cell-type specific weights per gene, the normalized matrices for counts-based sequencing data, such as single-cell RNA-sequencing, and bulk data, and the distribution mean, such as the Gaussian mean, and variances estimates for the counts-based sequencing, such as single-cell RNA-sequencing, and bulk data. For machine learning problems, selecting a proper loss function is important to the parameter estimation. The loss function is central to modern machine learning. The loss function takes an algorithm from theoretical to practical and transforms neural networks from glorified matrix multiplication into deep learning. At its core, a loss function is simple: it is a method of evaluating how well an algorithm models a dataset. If predictions are totally off, the loss function will output a higher number. If predictions are good, the loss function will output a lower number. As portions of the algorithm are revised in an attempt to improve the model, the loss function will advise whether the revisions are tending toward success.

Consider a few of the most popular loss functions currently being used, from simple to more complex. Mean Squared Error (MSE) is a basic loss function that is easy to understand and implement and generally works reasonably well. To calculate MSE, the difference between predictions and the ground truth is calculated, squared, and averaged across the whole dataset. Another loss function, the likelihood function, is also relatively simple and is commonly used in classification problems. The likelihood function takes the predicted probability for each input example and multiplies them. Although the output cannot be interpreted by human beings, the likelihood function is useful for comparing models. Log loss is a loss function also used frequently in classification problems and is a modification of the likelihood function with logarithms. Loss functions provide more than just a static representation of how a model is performing; they advise how the algorithm fits data in the first place. Most machine learning algorithms use some sort of loss function in the process of optimization, or finding the best parameters (weights) for a set of data.

The algorithm of the disclosed methods is designed to find the best set of proportion parameters by minimizing the difference between the mixture distribution of single cell data and that of the bulk cell data. FIG. 7 illustrates a comparison between the two distributions; one goal is to minimize the difference between the sum of the single cell data and the bulk cell data. Therefore, in some embodiments, the disclosed methods use the Kullback-Leibler (KL) divergence as its loss function (see, S. Kullback & R. Leibler, On Information and Sufficiency (Ann. Math. Stat. 1951)). KL-divergence is especially suitable for quantifying the similarity between two distributions. Let f₁(x) and f₂(x) be two probability density functions for a continuous variable X. The KL-divergence between the two is defined as:

$${D}_{KL} ( {f}_{1} \vert {f}_{2} ) = {\int }_{- ∞}^{∞} {f}_{1} ( x ) log \frac{{f}_{1} ( x )}{{f}_{2} ( x )} dx .$$

Next described are the model specifications in the implementation of the illustrative embodiments of the disclosed methods. The disclosed methods use the variable Y to represent the normalized expression value. One goal of the model is to estimate the proportion θₖ of cell type k in the bulk tissue. For a gene i in cell type k, the single cell expression (S) is

Yᵢₖ^{(S)}˜Gaussian(μᵢₖ^{(S)},σᵢₖ^{S(S)}),

and the same gene i in bulk data (B) is

Yᵢ^{(B)}˜Gaussian(μᵢ^{(B)},σᵢ^{B(B)}),

The probability densities are f^{S}(Yᵢ^{(S)}) and f^{B}(Yᵢ^{(B)}), respectively. The probability density in cell type k is written as f^{S}(Yᵢₖ^{(S)}). The loss function for gene i is

$${L}_{i} ( θ ) = {D}_{KL} 〈 {f}^{B} ( {Y}_{i}^{( B )} ) \vert {f}^{2} ( {Y}_{ik}^{( z )} ) 〉 , \\ where$$

$${f}^{\text{?}} ( {Y}_{i}^{( \text{?} )} ) = {\sum }_{\text{?} = \text{?}}^{K} {θ}_{k} {W}_{\text{?} k} {f}_{k}^{\text{?}} ( {Y}_{\text{?} k}^{( \text{?} )} ) , \\ \text{?} \text{indicates text missing or illegible when filed}$$

Assume that n highly variable genes are selected in Step 1, the total loss taking account of all genes is

L(θ)=Σᵢ^{m}Lᵢ(θ)ₖ

The defined loss function between the real bulk data distribution and the mixture distribution of single cells is the output from the fifth step of the disclosed methods. The proportions of single cells are set as unknown parameters in the loss function, which will be estimated in the next step.

Although possible, parameterizing the μ's and σ's would complicate the model unnecessarily because the goal of the disclosed method is to estimate θ's. The algorithm finds the set of global proportion parameters θ's that fit across all selected genes. A rough estimation of μ's and σ's from bulk and single-cell data suffices for the algorithm to find the best θ's, considering that the errors of estimation across all genes will be randomized and have a negligible global effect on the estimation of θ's. Therefore, the disclosed model directly uses the estimated μ's and σ's from Steps 3 and 4, and treats them as known parameters to compute the probability densities. The θ's will be the only unknown parameters to estimate in the model.

### Step 6: Model Estimation

The defined loss function between the real bulk data distribution and the mixture distribution of single cells output from the fifth step of the disclosed methods is the input for the sixth step. The methods adopt gradient descent to estimate the proportion parameters. Gradient descent is a first-order iterative optimization algorithm for finding the minimum of a function. To find a local minimum of a function using gradient descent, one takes steps proportional to the negative of the gradient (or approximate gradient) of the function at the current point. If, instead, one takes steps proportional to the positive of the gradient, one approaches a local maximum of that function; the procedure is then known as gradient ascent. Gradient descent was originally proposed by M. Cauchy in 1847 (see, M. Cauchy, Méthode Générale Pour la Résolution des Systèmes D'équations Simultanées (Úbersetzt von Richard Pulskamp 2010)). Compte rendu des séances l'académie des Sci. (1847). See also D. Bertsekas, Nonlinear Programming (2d Athena Scientific 1999).

The disclosed methods first derive the gradient (denoted as G) by taking the derivative of the loss function with respect to each θₖ. To simplify inference, the KL-divergence is decomposed into terms of entropy and cross-entropy,

$${D}_{KL} ( {f}^{B} \vert {f}^{s} ( \text{?} ) ) = \int {f}^{B} log ( {f}^{B} ) - \int {f}^{B} log ( {f}^{s} ( θ ) ) , \\ \text{?} \text{indicates text missing or illegible when filed}$$

Here θ represents the whole set of θₖ's. The parameters only exist in the second term (i.e., cross entropy). The first derivative with respect to θₖ is

$${G}_{k} = {∇}_{{θ}_{\text{?}}} {D}_{KL} 〈 {f}^{B} \vert {f}^{S} ( θ ) 〉 = - \int {f}^{B} \cdot d ( log ( {\sum }_{k = 1}^{K} {θ}_{k} {W}_{tk} {f}_{c}^{B} ) ) d ( {θ}_{\text{?}} ) = - \int {f}^{B} \cdot \frac{\text{?}}{\sum \text{?} {θ}_{k} {W}_{tk} f \text{?}} . \\ \text{?} \text{indicates text missing or illegible when filed}$$

The computation of Gₖ involves numeric integration over the support of normalized expression values. In theory, Y∈R. That means the computation of integration can be very slow. To speed up the algorithm, the methods limit the support within the 99% quantile region. The methods further replace the integration with the discrete approximation,

$$\tilde{{G}_{k}} = - {\sum }_{i = 1}^{τ} {f}^{B} \cdot \frac{{f}_{k}^{\text{?}}}{{\sum }_{k = 1}^{K} {θ}_{k} {W}_{tk} {f}_{k}^{\text{?}}} . \\ \text{?} \text{indicates text missing or illegible when filed}$$

where T is the number of points sampled from the 99% quantile region of normalized expression values. The larger T is, the more accurate the approximation is. It has been found that T=100 is a sufficient number to achieve reasonable accuracy while achieving a 100-fold increase in the speed of the algorithm.

To run gradient decent, the methods randomly initialize non-negative θ with θ [symbol] , then simultaneously update all θₖ's at each step such that

θₖ^{t+L}=θₖ^{t}−α·

[symbol]

where α is the learning rate. Because the methods are estimating proportions, at each update the method rescales θₖ's such that they add up to 1,

$${θ}_{k}^{\text{?} + 1} = \frac{{θ}_{k}^{\text{?} + 1}}{{\sum }_{\text{?} = 1}^{K} {θ}_{k}^{\text{?} + 1}} . \\ \text{?} \text{indicates text missing or illegible when filed}$$

The methods define the convergence as ∥θ^{t+1}−θ^{t}∥₂<0.0006, and set α−0.3 for satisfactory accuracy and a reasonable convergence rate.

In some embodiments, the counts-based sequencing data is single-cell RNA-sequencing data; wherein the counts-based sequencing counts is single-cell RNA-sequencing counts; and wherein the counts-based sequencing data matrix is single-cell RNA-sequencing data matrix. In some embodiments, the counts-based sequencing data is ATAC-seq data; wherein the counts-based sequencing counts is ATAC-seq counts; and wherein the counts-based sequencing data matrix is ATAC-seq data matrix.

In some embodiments, the cross-sample distribution for each cell type and for each of the subset of the most variably expressed genes from the counts-based sequencing data matrix is a cross-sample Gaussian distribution. In some embodiments, the bulk distribution for each subset of the most variably expressed genes from a normalized bulk matrix and a subset of the most variably expressed genes is a bulk Gaussian distribution.

In some embodiments, the step of selecting comprises calculating a standard deviation for each gene within the plurality of genes, determining a threshold standard deviation number, and selecting the subset of most variably expressed genes having a standard deviation above that threshold number. In some embodiments, the step of computing cell-type specific weights comprises comparing the total mean variance to the within-cell type mean variance for each of a fixed number of cells. In some embodiments, the step of fitting comprises using the whole distribution when estimating the mixing proportion. In some embodiments, the step of fitting further comprises obtaining the distribution by fitting the normalized count to the distribution and estimating the variance and mean for each gene. In some embodiments, the distribution is a Gaussian distribution. In some embodiments, the step of defining a loss function comprises applying Kullback-Leibler divergence. In some embodiments, the step of applying the loss function comprises adopting gradient descent.

The present disclosure also provides another embodiment of AdRoit methods for deconvolving bulk RNA-sequencing data. The embodiments of the disclosed methods discussed herein may be described as “accurate and robust methods for the inference of transcriptome composition” and identified by the acronym “AdRoit.” The AdRoit methods aim to accurately and robustly estimate the proportion of cell types from compound transcriptome data including bulk RNA-seq and spatial transcriptome data. The methods utilize as a reference the relevant pre-existing single cell RNA-seq data with cell identity annotation, select informative genes, and estimate the expression mean and dispersion of the selected genes per cell type. Further, in one embodiment, the AdRoit methods calculate the gene-wise variability across samples, as well as their cell type specificity, according to which the loss function of each gene will be weighted differently in the model. Still further, the AdRoit methods compute a gene-wise scaling factor to minimize the technology difference between single cell and the target compound data. Together, the AdRoit methods feed them into a regularized model where the cell type percentages are estimated by optimizing a weighted sum of the loss functions per gene. The keys to the accuracy and robustness of the methods include 1) selecting most informative genes used for the deconvolution task; 2) properly weighted per-gene loss functions by how specifically they can differ one cell type from the rest, and by how stable their expressions are across multiple samples; 3) gene-wise scaling factors to normalize the gene expression values from different sequencing platforms (e.g. TPM or read counts from bulk RNA-seq, unique molecular identifier (UMI) from single cell RNA-seq and spatial transcriptome sequencing); and 4) a regularized regression model that avoids collinearity between closely related cell types (e.g., subtypes).

In some embodiments, the AdRoit methods for deconvolving bulk or spatial RNA-sequencing data comprise any one or more of the following exemplary steps: i) obtaining input from three sources (bulk or spatial RNA-seq data, single cell RNA-seq data, and cell type annotations) and selecting a subset of the most variably expressed genes from a matrix of counts-based single cell sequencing data, wherein the matrix of counts-based sequencing data comprises counts-based sequencing counts against each gene within a plurality of genes for a fixed number of cells; ii) estimating the mean and dispersion parameters of the expression per gene per cell type; iii) computing the cross cell type specificity of genes; iv) within each cell type, for each gene, estimating cross-sample gene expression variability based on the average gene expression of multiple cells in each sample depending on multi-sample availability or creating multiple samples by subsampling cells from the same sample; v) estimating gene-wise scaling factors using both compound data and single cell data; and vi) building a weighted and regularized regression model using all of the known quantities and using the model to estimate cell type proportions in the bulk or spatial RNA-sequencing data; thereby inferring the percentage of cell types in the bulk or spatial RNA-sequencing data. These steps are illustrated in FIG. 11, which shows an overview of the workflow of one embodiment of the disclosed methods. Each of the steps is discussed below, in turn, with reference to the input, output, and purpose or rationale for each step. Each of these process steps can be carried out by a computing device, such as a computer. In some embodiments, all of the process steps are carried out by a computer. Spatial transcriptomes is a special type of bulk sequencing with very few cells.

In some embodiments, the methods comprise the first step. In some embodiments, the methods comprise the first step and one or more of the second, third, fourth, fifth, and sixth steps, or any combination of these additional steps. In some embodiments, the methods comprise the second step. In some embodiments, the methods comprise the second step and one or more of the first, third, fourth, fifth, and sixth steps, or any combination of these additional steps. In some embodiments, the methods comprise the third step. In some embodiments, the methods comprise the third step and one or more of the first, second, fourth, fifth, and sixth steps, or any combination of these additional steps. In some embodiments, the methods comprise the fourth step. In some embodiments, the methods comprise the fourth step and one or more of the first, second, third, fifth, and sixth steps, or any combination of these additional steps. In some embodiments, the methods comprise the fifth step. In some embodiments, the methods comprise the fifth step and one or more of the first, second, third, fourth, and sixth steps, or any combination of these additional steps. In some embodiments, the methods comprise the sixth step. In some embodiments, the methods comprise the sixth step and one or more of the first, second, third, fourth, and fifth steps, or any combination of these additional steps.

In some embodiments, the AdRoit methods for deconvolving bulk or spatial RNA-sequencing data comprise any one or more of the following exemplary steps: i) computing the cross cell type specificity of genes; ii) within each cell type, for each gene, estimating cross-sample gene expression variability based on the average gene expression of multiple cells in each sample depending on multi-sample availability or creating multiple samples by subsampling cells from the same sample; iii) estimating gene-wise scaling factors using both compound data and single cell data; and iv) building a weighted and regularized regression model using all of the known quantities and using the model to estimate cell type proportions in the bulk or spatial RNA-sequencing data; thereby inferring the percentage of cell types in the bulk or spatial RNA-sequencing data. Each of the steps is discussed below, in turn, with reference to the input, output, and purpose or rationale for each step. Each of these process steps can be carried out by a computing device, such as a computer. In some embodiments, all of the process steps are carried out by a computer. Spatial transcriptomes is a special type of bulk sequencing with very few cells. In some embodiments, computing the cross cell type specificity of genes is carried out based upon estimated mean and dispersion parameters of the expression per gene per cell type from a subset of the most variably expressed genes selected from a matrix of counts-based single cell sequencing data (obtained from three sources: i) bulk or spatial RNA-seq data, ii) single cell RNA-seq data, and iii) cell type annotations) wherein the matrix of counts-based sequencing data comprises counts-based sequencing counts against each gene within a plurality of genes for a fixed number of cells.

In some embodiments, the methods comprise the first step. In some embodiments, the methods comprise the first step and one or more of the second, third, and fourth steps, or any combination of these additional steps. In some embodiments, the methods comprise the second step. In some embodiments, the methods comprise the second step and one or more of the first, third, or fourth steps, or any combination of these additional steps. In some embodiments, the methods comprise the third step. In some embodiments, the methods comprise the third step and one or more of the first, second, and fourth steps, or any combination of these additional steps. In some embodiments, the methods comprise the fourth step. In some embodiments, the methods comprise the fourth step and one or more of the first, second, and third steps, or any combination of these additional steps.

### Step 1: Select Genes

A purpose of the first step in the second embodiment of the disclosed methods is to select the most informative genes. This step is applied to single-cell RNA-seq (scRNA-seq) data, but can be applied to any counts-based sequencing data as set forth herein. The step begins by obtaining input from three sources: bulk or spatial RNA-seq data, single cell RNA-seq data, and cell type annotations. Thus, the input data are a single cell UMI count matrix with cell type annotations associated with each cell. Each column of the matrix corresponds to a cell and each row of the matrix to a gene. Each entry in the matrix is the UMI count for a particular gene in a cell. The bulk data to be deconvolved can be either transcripts per kilobase million (TPM) or read counts. Each row of the matrix is a gene and each column of the matrix is a sample. The spatial transcriptome data to be deconvolved is also a UMI count matrix, but each column of the matrix is a spatial spot and each row of the matrix is a gene. The mathematic notations and terms used in describing the steps of the methods are defined as follows:

i—index of gene, i=1, . . . , I

k—index of cell type, k=1, . . . , K

kⁱ—the index of cell type that has the highest mean expression of gene i

j—index of samples (bulk RNA-seq) or spatial spots

nₖ—number of cells in cell type k

Xᵢₖ—the set of single cell UMI counts of gene i for all cells in cell type k

Yᵢⱼ—counts of gene i in bulk sample or spatial spot j

λᵢₖ—dispersion parameter for gene i in cell type k

pᵢₖ—probability for gene i in cell type k getting one UMI

λᵢⱼ—dispersion parameter for gene i in bulk sample or spatial spot j

pᵢⱼ—probability for gene i in cell type in bulk sample or spatial spot j

μᵢₖ—mean expression of gene i in cell type k

σᵢₖ²—variance of the expression of gene i in cell type k

wᵢ^{C}—cell type specificity weight for gene i

μᵢ²—mean of gene i in the replicated or bootstrapped bulk samples or spatial spots

(σᵢ²)³—variance of gene i in replicated or bootstrapped bulk samples or spatial spots

wᵢ^{S}—cross-sample variability weight for gene i,

τₖ—rough estimates of the percentage of cell type k

rᵢ—adaptively learned scaling factor for gene i

βₖ—unscaled regression coefficient for cell type k

Gₖ—gradient function with respect to βₖ

θₖ—final estimates of the percentage of cell type k

\hat{·}—estimated quantities from model fitting

MLE—maximum likelihood estimation

VMR—variance-to-mean ratio

NB( )—negative binomial distribution

LH( )—likelihood function

L( )—loss function

Using the input data, the first step in the second embodiment of the disclosed methods selects a subset of the most variably expressed genes from a normalized matrix of counts-based sequencing data. The matrix of counts-based sequencing data comprises counts-based sequencing counts against each gene within a plurality of genes for a fixed number of cells. A step to successfully deconvolving cell type composition is selecting the proper set of genes. The methods select genes that contains important information to differentiate cell types, excluding non-informative genes that potentially introduce noise.

As illustrated in FIG. 12A, the methods select genes in one of two alternative options. The first option is to use the union of the genes whose expression is enriched in each cell type in the single cell UMI count matrix. These genes are referred to as marker genes. The second option is to use the union of the genes that vary the most across all the cells in the single cell UMI count matrix. These genes are referred to as the highly variable genes. This second option computes the variances for each gene after cell number balancing and variance stabilizing transformation (VST) normalization, then selects genes with the highest variances. Either option yields comparably accurate estimations.

To select the marker genes, either a predefined marker gene list can be input or a build-in tool can be used. The build-in tool takes as input the single cell UMI count matrix and cell type annotations. For each cell type, the tool computes the fold change between the average UMI in that cell type and the average UMI in all other cell types, then ranks genes by descending fold changes. Selection of approximately the top 200 genes from each cell type would be sufficient to resolve complex compound transcriptome data. Because some genes may mark more than one cell type, selected markers presenting in no more than five cell types are desired to ensure specificity. Alternately, selected markers presenting in no more than a fixed number of cell types or a fraction of total number of cell types, whichever is smaller to ensure specificity. Selection of a minimum of about 1,000 total unique genes from the union of the marker genes of all cell types is desired to ensure an accurate estimation.

Finding marker genes can sometimes be time-consuming and require extensive computational resources. If marker genes are not immediately available, however, the methods can select the highly variable genes. Usually these genes are also informative to differentiate cell types. To avoid the risk that the selected highly variable genes might be dominated by large cell clusters while underrepresenting small clusters, the cell types in the single cell UMI count matrix can be balanced by finding the median size of all the cell clusters. Then cells from each cluster can be sampled to make them equal to this size. Next, the methods compute the variance of each gene across the cells in the balanced single cell UMI matrix. Given the well-known over-dispersed nature in RNA-seq data, directly computing variances from a count matrix can be prone to error. Therefore, the methods compute variances on the normalized data by variance stabilization transformed (VST). See Anders, S. & Huber, W., “Differential expression analysis for sequence count data,” Genome Biol. (2010). Genes with the top 2,000 large variances can be selected. The algorithm of selecting highly variable genes is the same as programed in the “Seurat” R package disclosed in A. Butler, Nat. Biotechnol.

FIG. 12B provides a hypothetical example illustrating the type of cells that can be selected. Four graphs are shown, one graph for each of four genes, with each graph reflecting gene expression versus cell type. In the example depicted, Gene 3 exhibits good variation across cell types, and Gene 4 is a strong marker for cell type C1. Either of these genes will have good information about the variety of the cell types and, therefore, can be selected for modeling.

### Step 2: Estimate Gene Mean & Dispersion Per Cell Type

Modeling single cell RNA-seq data can be challenging due to the cellular heterogeneity and technical sensitivity and noise. Although the expression of some genes cannot be detected by chance, other genes may be found to be highly dispersed. The dispersed genes can lead to excessive variability even within the same cell type. In addition, due to an increasingly large number of cells per study, directly estimating cell percentages using every cell as a training sample is computationally challenging. The disclosed methods combat high noise and computational complexity by aggregating individual cells at the cell type level. The mean and dispersion of each gene per cell type can be estimated. This strategy reduces the data complexity while preserving the cell type specific information.

Although typical analyses of RNA-seq data start with normalization, the disclosed methods do not normalize before estimating the mean. Performing a normalization across all cell types forces every cell type to have the same amount of RNA transcripts, measured by the total UMI counts per cell. But different cell types can have dramatically different amounts of transcripts. For example, the amount of RNA transcripts in neuronal cells is about ten times of the amount in glial cells. Thus, normalization can falsely alter the relative abundance of cell types, misleading the estimation of cell type percentages. To avoid this problem, the disclosed methods model the means using the raw UMI counts.

Studies have shown that counts of UMIs follow a negative binomial distribution. See Hafemeister, C. & Satija, R., “Normalization and variance stabilization of single-cell RNA-seq data using regularized negative binomial regression,” Genome Biol. (2019); and Svensson, V., “Droplet scRNA-seq is not zero-inflated,” Nature Biotechnology (2020). Therefore, the disclosed methods fit negative binomial distributions to single cells of each cell type. FIG. 13 illustrates the step of estimating the mean and dispersion parameters by fitting a negative binomial distribution for each gene in cell type k. The disclosed methods later build a model based on the estimated mean and dispersion parameters from the selected genes. More specifically, let Xᵢₖ be the set of single cell UMI counts of gene i∈1, . . . , I for all cells in cell type k∈1, . . . , K. The letter I denotes the number of selected genes, and K denotes the number of cell types in the single cell reference. The distribution of Xᵢₖ follows a negative binomial distribution,

Xᵢₖ˜NE(λᵢₖ,μᵢₖ),

where λᵢₖ is the dispersion parameter of the gene i in cell type k, and pᵢₖ is the success probability, i.e., the probability of gene i in cell type k getting one UMI. The two parameters are estimated by maximum likelihood estimation (MLE). The likelihood function is

$$LH ( {λ}_{ik} , {p}_{ik} \vert {X}_{ik} ) = {\prod }_{\text{?}}^{{n}_{k}} f ( {\text{?}}_{ik} \vert {λ}_{ik} , {p}_{ik} )$$

$$\text{?} \text{indicates text missing or illegible when filed}$$

where nₖ is the number of cells in cell type k, and f is the probability mass function of the negative binomial distribution. The MLE estimates are then given by

$$( ) = {argmin}_{{λ}_{ik} , {p}_{ik}} LH ( {λ}_{ik} , {p}_{ik} \vert {X}_{ik} ) .$$

Once the success probability and the dispersion are estimated, the mean estimates can be computed numerically according to the properties of a negative binomial distribution:

$${μ}_{ik} = \frac{1 -}{} , and {σ}_{ik}^{2} = \frac{{( 1 - )}^{2}}{} .$$

Estimation using MLE has been readily coded in many R packages. R is a programming language and free software environment for statistical computing and graphics supported by the R Foundation for Statistical Computing. Suitable is the “fitdist( )” function from the “fitdistrplus” package, which offers fast computation speed and flexibility in selecting distributions. In the disclosed methods, estimations are carried out for each selected gene in each cell type, resulting in an matrix of cell type means.

### Step 3: Compute the Cross Cell Type Specificity of Genes

Genes with cell type specific expression patterns better represent particular cell types and, therefore, are more important when used to resolve cell type composition. In line with this property, the disclosed methods weight genes with high specificity more than less specific genes. Highly specific genes usually have consistently high expression and relatively low variance among cells within a cell type. To compute the cell type specificity of a gene, the disclosed methods first identify the cell type in which the gene has the highest expression (i.e., most specifically expressed cell type), then define the specificity of this gene as the mean-to-variance ratio within the cell type. A high ratio assigns a high weight to the gene in the model described later. The disclosed methods use the estimated mean and variance parameters from the negative binomial fitting from step 2 (μᵢₖ and σᵢₖ² in the equations above). Let k′ be the index of the cell type that has the highest mean expression of gene i, and

$${k}^{′} = {argmax}_{k} \{ {μ}_{ik} \vert k ∈ 1 … K \}$$

then the cell type specificity weight for gene i, denoting wᵢ^{S}, is given by

$${w}_{i}^{S} = \frac{{μ}_{{ik}^{′}}}{{σ}_{{ik}^{′}}^{2}} .$$

The cell type specificity weight is computed for each gene in the set of selected genes. Two parameters are used here (average gene expression level and variance within each cell type), but only the average gene expression level is used to compare across different cell types without specifying the criteria as being cell type specific. For instance, the expression is at least x-fold higher in the highest expression cell type than the second or the average of the rest cell types.

### Step 4: Estimate Cross-Sample Gene Variability

The variability of a gene indicates how stable a gene is across samples. The concept of weighting genes based on variability across samples is reported in the article by Wang et al. (supra). Wang et al. defined variability as the cross-sample variance. By weighting down the high variability genes, the authors achieved a great advantage over the traditional unweighted methods. Genes with low cross-sample variability better represent the population, hence are more trustworthy when used to learn the cell composition. The disclosed methods incorporate the similar concept to weight the importance of genes. The disclosed methods define the variability in a different and more superior way. Specifically, the disclosed methods use the variance-to-mean ratio (VMR) to define the cross-sample gene variability. Here the mean and variance are computed across samples. The VMR is better scaled than the simple variance, and it can avoid both underweighting genes that have low expression and overweighting genes that are unstable.

In addition, the disclosed methods can be extended to address applications in which multiple samples are not available. Three options are available to compute the VMR, depending on whether multi-sample data are available. Typically, the compound transcriptome data to be deconvolved have multiple samples. In bulk RNA-seq data, multiple samples are usually included to control for biological variability. In spatial transcriptome data, the neighbor spatial dots can be seen as multiple samples. Therefore, in the first option, the disclosed methods compute the cross-sample gene variability from compound transcriptome data. In the second option, the compound data do not have multi-samples whereas the single cell data do and the disclosed methods synthesize multiple compound samples, each of which is an average of all cells belonging to one of the samples in the single cell reference. Finally, if multi-samples are unavailable for both types of data, in the third option the disclosed methods repetitively bootstrap the single cells and average the sampled cells to make multiple, synthesized compound samples.

After the multiple compound samples are obtained, let Ali denote the counts of sequences for gene i in sample j∈1, . . . , J, then

Yᵢⱼ˜NB(λᵢⱼ,pᵢⱼ),

where λᵢⱼ is the dispersion parameter of the gene i in the compound sample j, and pᵢⱼ is the success probability. Again, the disclosed methods use MLE to get the estimates [symbol] and [symbol] , following which cross-sample mean and variance can be numerically computed:

$${μ}_{i}^{2} = \frac{1 - }{} , {( {σ}_{i}^{2} )}^{2} = \frac{{( 1 - )}^{2}}{} ,$$

and cross-sample variability for gene i is then defined as

$${VMR}_{i} - \frac{{( {σ}_{i}^{2} )}^{S}}{{μ}_{i}^{2}} - \frac{1}{{w}_{i}^{C}} ,$$

where wᵢ^{C} is later used in the model. The cross-sample variability weight is computed for each gene in the set of selected genes.

### Step 5: Estimate Gene-Wise Scaling Factors

When linking the compound data to the single cell data, rescaling factors are often used to account for the library size and platform difference. The existing methods all adopt a single rescaling factor for each unit of sample, i.e., all genes of a single sample are multiplied by the same factor (see, Wang et al. (supra) and Andersson et al., “Spatial mapping of cell types by integration of transcriptomics data,” bioRxiv (2019)). This operation is based on the assumption that the impact of platform difference on every gene is the same and linearly scaled among different cell types, which is hardly true. In addition, because estimates can be easily affected by outliers in a linear model, estimation of cell proportions can be steered away from the truth by extremely high outlier genes. Therefore, applying a uniform scaling factor to all genes is inappropriate.

To overcome this problem, the disclosed methods instead estimate gene-wise scaling factors via an adaptive learning strategy and rescale each gene with its respective scaling factor. To proceed, the disclosed methods first input the mean gene expression from the compound samples (μᵢ^{S} from step 4 above) and the estimated means of each cell type from the single cell data (μᵢₖ from step 2 above), then apply a traditional non-negative least square regression (NNLS) to get a rough estimation of the proportions of each cell type, denoted τₖ. See Chen, D. & Plemmons, R., “Nonnegativity constraints in numerical analysis,” in The Birth of Numerical Analysis (2009). For each gene, a predicted mean expression (Σₖ^{K} [symbol] μᵢₖ in the equation below) is computed as the weighted sum of the means of each cell type wherein the weights are the roughly estimated proportions. The regression equation is given by

μᵢ^{S}=A·(Σₖ^{K}τₖμᵢₖ+s),0≤τₖ,Σₖ^{K}τₖ=1

where A is a constant to ensure τₖ's sum to 1 and s is the error term. The disclosed methods use the “nnls( )” function in the package “nnls” to estimate τₖ's. Next, the disclosed methods calculate the ratio between the mean expression from the compound samples and the predicted means, and define the gene-wise rescaling factor as the logarithm of the ratio plus 1:

$${\text{?}}_{t} = log ( \frac{{μ}_{t}^{2}}{{\sum }_{k}^{K} \hat{{τ}_{k}} {μ}_{\text{?}}} + 1 ) . \\ \text{?} \text{indicates text missing or illegible when filed}$$

Given the dispersion property of data, the logarithm of the ratio is a more appropriate statistic as it results in relatively stable scaling factors. The addition of 1 avoids taking the logarithm on zero. By multiplying the flexible gene-wise rescaling factor, the “outlier” genes will be pushed toward the true regression line while the genes around the true regression line are less affected.

FIG. 14 provides a hypothetical example demonstrating the effect of the gene-wise scaling factor. Ideally, an accurate estimation of slope (i.e., cell percentage) would be the slope of the left-most line in FIG. 14. A direct fitting would result in the right-most line, however, due to the impact of the outlier genes. Outlier genes can be induced because the platform difference affects genes differently. The disclosed methods adopt an adaptive learning approach that first learns a rough estimation of the slope (i.e., the right-most line), then moves the outlier genes toward it such that the more deviated genes will be moved more toward the true line (i.e., along the longer arrows). After this adjustment, the new estimated slope (the center line) is closer to the true line (the left-most line) and, therefore, is a more accurate estimation.

### Step 6: Build a Weighted and Regularized Regression Model

In the sixth step, the disclosed methods build a model that incorporates all of the above factors to do the actual estimation of cell percentages. The methods build upon a non-negative least square regression model, and give high weights to the genes with high cell type specificity and low cross-sample variability. This step is carried out by optimizing a weighted sum of squared loss function L, where the weights consist of two components: wᵢ^{S} from step 3 above and wᵢ^{C} from step 4 above. The gene-wise scaling factor tailored for each gene minimizes the technology difference between compound sample and single cell data (rᵢ from step 5 above).

In cases of complex tissues (e.g., neural tissues) where many highly similar subtypes are common, closely related subtypes can have strong collinearity, leading to overestimation of some cell types while underestimating or even missing others. The disclosed methods address this problem by including a L2 norm of the estimates as the regularization component. Denote βₖ as the unscaled coefficient for cell type k. For a compound transcriptome sample j, the loss function is given by

$${L}_{1} ( {β}_{1} , … , {β}_{k} \vert {y}_{11} , {ω}_{1}^{c} , {ω}_{1}^{2} , {r}_{1} , \text{?} ) = {\sum }_{1}^{i} {ω}_{1}^{c} \cdot {ω}_{1}^{2} \cdot {( {y}_{N} - \text{?} {\sum }_{k}^{k} {β}_{k} )}^{2} + {\sum }_{k}^{k} {β}_{k}^{2} . \\ \text{?} \text{indicates text missing or illegible when filed}$$

Then the coefficient βₖ can be estimated by minimizing the loss function with the constraint β₁, . . . , β_{K}≥0,

$$, … , = {{argmax}_{{β}_{1} , {…β}_{K} > {o}^{{L}_{l}}}}_{\text{?}} . \\ \text{?} \text{indicates text missing or illegible when filed}$$

The estimation is done by a gradient projection method disclosed by Byrd et al., “A Limited Memory Algorithm for Bound Constrained Optimization,” SIAM J. Sci. Comput. (1995). The gradient function is derived by taking the partial derivative of the loss function with respect to βₖ,

$${G}_{k} = {∇}_{{β}_{k}} {L}_{l} = - 2 {\sum }_{l}^{L} {r}_{i} \cdot \cdot {w}_{i}^{\text{?}} \cdot {w}_{i}^{2} \cdot ( {y}_{il} - {r}_{i} \cdot {\sum }_{k}^{K} {β}_{k} ) + 2 {β}_{k} . \\ \text{?} \text{indicates text missing or illegible when filed}$$

The disclosed methods use the function “optim( )” from the R package “stats” to do the estimation, providing the loss function and the gradient function above. To get the final estimates of cell type proportions, the disclosed methods rescale the coefficients βₖ's to ensure a summation of 1,

$${θ}_{k} = \frac{{\sum }_{k}^{K} }{} .$$

Each compound sample j is independently estimated by the above described model.

In some embodiments, the plurality of genes from the normalized matrix of counts-based sequencing data comprises at least about 20,000 genes. In some embodiments, the selected subset of the most variably expressed genes comprises from about 1,000 to about 5,000 genes. In some embodiments, the selected subset of the most variably expressed genes comprises about 2500 genes.

In some embodiments, any of the methods described herein can further comprise identifying the proportion of RNA from each cell type from which the bulk or spatial RNA-sequencing data were obtained. In some embodiments, any of the methods described herein can further comprise identifying the proportion of each cell type from which the bulk or spatial RNA-sequencing data were obtained. In some embodiments, any of the methods described herein can further comprise identifying the proportion of RNA from each cell type from which the bulk or spatial RNA-sequencing data were obtained. In some embodiments, any of the methods described herein can further comprise identifying the proportion of each cell type from which the bulk or spatial RNA-sequencing data were obtained.

The methods of deconvolution of bulk or spatial RNA sequencing data using information from counts-based sequencing data, such as scRNA-seq data, can be used in a variety of ways. In general, the methods described herein provide a more robust and accurate estimate of a specific cell type within a population of a plurality of cell types. In addition, the methods described herein can be applied to all counts-based sequencing data (i.e., the methods described herein are not limited to scRNA-seq data but can apply to other types of count-based sequencing data such as ATAC-seq, to cellular products other than RNA, and to a wide variety of mixture samples such as mixtures of different tissues).

The methods described herein can be used, for example, to estimate the mixture proportion for one or more particular cell types given a gene expression pattern of single cell types. A bulk tissue is usually composed of multiple cell types with different proportions. Using the liver as an example, there exist hepatocyte cells, stellate fat storing cells, Kupffer cells, and endothelial cells. The methods described herein can be used to estimate the proportion of these individual cell types in the bulk liver tissue. The mixture proportion for one or more particular cell types can be determined, for example, for an organ, tissue, cell culture, or the like.

The methods described herein can also be used, for example, to detect tissue contamination. For example, a biopsy or other tissue sample obtained from a human may have a desired first cell type within the biopsy but may also have, or be suspected of having, a second undesirable cell type. The methods described herein can be used to determine whether the biopsy or tissue sample is contaminated with the second cell type and, if so, the amount of contamination. To give an example, muscle contamination is often seen in the RNA-seq data from heart tissue. The methods described herein can be used to determine whether the heart tissue is contaminated by the muscle cells during dissection and isolation, and to estimate how many muscle cells exist in the heart tissue samples.

The methods described herein can also be used, for example, to detect tumor infiltration. For example, a biopsy or other tissue sample can be obtained from a particular tumor within a human and the presence of, identity of, and/or extent of infiltration of the tumor with non-tumor cells can be determined using the methods described herein. In some embodiments, the non-tumor cells infiltrating the tumor are immune cells, such as, for example, macrophages, lymphocytes, and natural killer cells. In some embodiments, the lymphocytes are B lymphocytes and/or T lymphocytes. In some embodiments, the lymphocytes are tumor infiltrating lymphocytes (TILs). A tumor that has been infiltrated with immune cells, such as T lymphocytes, is a “hot” tumor. A tumor that contains a few or no infiltrating T lymphocytes, and thus is not recognized and does not provoke a strong response by the immune system, is a “cold” tumor. Thus, the methods disclosed herein can be used to determine whether a particular tumor within a human is considered, such as by a health practitioner, to be a hot tumor or a cold tumor, by determining the presence of, identity of, and/or extent of infiltration of the tumor with immune cells. Hot tumors are more susceptible to immunotherapy than cold tumors. Thus, a human having a hot tumor would be a better candidate for immunotherapy than a human having a cold tumor. Hot and cold tumors are discussed in, for example, Galon et al., Nat. Rev. Drug Disc., 2019, 18, 197-218; Bonaventura et al., Front. Immunol., 2019, 10, 168, 1-10; and Seidel et al., Front. Oncol., 2018, 8, 86. Accordingly, the methods described herein can be used to stratify patients for their susceptibility to immunotherapy. The methods can also be used to estimate the proportions of infiltrating cells, such as immune cells, in a particular tumor—which can be used to identify immunotherapy susceptible patients. In some embodiments, the methods described herein further comprise administering immunotherapy to a human who has an infiltrated tumor.

In some embodiments, the cells from which the bulk RNA-sequencing data were obtained comprise tumor cells, and the method further comprises identifying the proportion of immune cells among the tumor cells. In some embodiments, the immune cells comprise tumor infiltrating lymphocytes. In some embodiments, the immune cells comprise CD8-positive T lymphocytes. In some embodiments, the immune cells comprise CD8-positive T lymphocytes and dendritic cells. In some embodiments, the methods described herein further comprise characterizing the tumor from which the tumor cells were obtained as a hot tumor or a cold tumor.

In some embodiments, the tumor is characterized as a hot tumor and is present in a subject, and the methods further comprise determining whether the subject has less than, equal to, or greater than a threshold level of infiltrating immune cells. In some embodiments, the immune cells comprise CD8-positive T lymphocytes. In some embodiments, the immune cells comprise CD8-positive T lymphocytes and dendritic cells. In some embodiments, the subject has greater than a threshold level of infiltrating immune cells, and the methods further comprise identifying the subject as a candidate for immunotherapy.

In some embodiments, the immunotherapy comprises adoptive cell therapy. In some embodiments, the adoptive cell therapy comprises chimeric antigen receptor T-cell (CAR T-cell) therapy. In some embodiments, the immunotherapy comprises an immune checkpoint inhibition therapy. In some embodiments, the immune checkpoint inhibition therapy comprises an antibody that blocks cytotoxic T-lymphocyte-associated antigen-4 (CTLA-4), an antibody that blocks programmed cell death protein 1 (PD-1), an antibody that blocks programmed cell death ligand 1 (PD-L1), or an antibody that blocks lymphocyte-associated gene 3 (LAG3), or any combination thereof. In some embodiments, the immune checkpoint inhibition therapy comprises an antibody that blocks cytotoxic T-lymphocyte-associated antigen-4 (CTLA-4), including, but not limited to, ipilimumab and REGN4659. In some embodiments, the immune checkpoint inhibition therapy comprises an antibody that blocks programmed cell death protein 1 (PD-1), including, but not limited to, nivolumab, pembrolizumab, and cemiplimab. In some embodiments, the immune checkpoint inhibition therapy comprises an antibody that blocks programmed cell death ligand 1 (PD-L1), including, but not limited to, atezolizumab. In some embodiments, the immune checkpoint inhibition therapy comprises an antibody that blocks lymphocyte-associated gene 3 (LAG3), including, but not limited to, REGN3767.

The methods described herein can also be used, for example, to characterize or score tumor microenvironment. For example, a biopsy or other tissue sample can be obtained from a particular tumor within a human and the presence of, identity of, and/or extent of infiltration of the tumor microenvironment with tumor microenvironment cells can be determined using the methods described herein. Tumor microenvironment cells include, but are not limited to, stromal cells and immune cells. Stromal cells include, but are not limited to, fibroblasts (such as cancer-associated fibroblasts), cancer-associated adipocytes, pericytes, and endothelial cells, such as lymphatic endothelial cells and blood vessel endothelial cells. Immune cells include, but are not limited to, macrophages, lymphocytes, and natural killer cells. In some embodiments, the lymphocytes are B lymphocytes and/or T lymphocytes. In some embodiments, the T lymphocytes are TILs.

A human who has a tumor microenvironment that has been infiltrated with such tumor microenvironment cells may be at a more advanced stage of the cancer than a tumor microenvironment that has not been infiltrated with such tumor microenvironment cells. Thus, the methods disclosed herein can be used to determine whether a particular tumor microenvironment within a human is considered, such as by a health practitioner, to be at an advanced stage of cancer, by determining the presence of, identity of, and/or extent of infiltration of the tumor microenvironment with tumor microenvironment cells. Accordingly, the methods described herein can be used to stratify patients for their susceptibility to immunotherapy based upon the cell type composition of the tumor microenvironment. The methods can also be used to estimate the proportions of infiltrating cells in a particular tumor microenvironment—which can be used to identify immunotherapy susceptible patients. In some embodiments, the methods described herein further comprise administering immunotherapy to a human who has an infiltrated tumor microenvironment.

In some embodiments, the cells from which the bulk RNA-sequencing data were obtained comprise tumor microenvironment cells, and the methods further comprise identifying the proportion of tumor cells among the tumor microenvironment cells. In some embodiments, the cells from which the bulk RNA-sequencing data were obtained comprise tumor microenvironment cells, and the methods further comprise identifying the proportion of immune cells among the tumor microenvironment cells. In some embodiments, the cells from which the bulk RNA-sequencing data were obtained comprise tumor microenvironment cells, and the methods further comprise identifying the proportion of cancer-associated fibroblasts among the tumor microenvironment cells. In some embodiments, the cells from which the bulk RNA-sequencing data were obtained comprise tumor microenvironment cells, and the methods further comprise identifying the proportion of cancer-associated adipocytes among the tumor microenvironment cells. In some embodiments, the cells from which the bulk RNA-sequencing data were obtained comprise tumor microenvironment cells, and the methods further comprise identifying the proportion of lymphatic endothelial cells among the tumor microenvironment cells. In some embodiments, the cells from which the bulk RNA-sequencing data were obtained comprise tumor microenvironment cells, and the methods further comprise identifying the proportion of blood vessel endothelial cells among the tumor microenvironment cells.

The methods described herein can also be used, for example, to estimate cell type proportions in samples of the islets of Langerhans, which are clusters of endocrine cells within the pancreas. Pancreatic islets contain five endocrine cell types (α, β, δ, ε, and γ) of which β cells secrete insulin and are gradually lost in humans having Type 2 diabetes. The “normal” population of β cells should be about 50-60%. Thus, determination of the cell type proportion of pancreatic islet cells by the methods described herein can be used to determine the presence of Type 2 diabetes and track the development and/or treatment thereof.

The methods described herein can also be used, for example, to estimate cell type proportions in samples of kidney cells to detect kidney diseases such as, for example, chronic kidney disease (CKD), characterized by the gradual loss of kidney function. Fibrosis is the histologic hallmark common to all CKD models. In addition to neutrophils and podocytes, kidney cells fall into two large groups: immune cell types (macrophages, fibroblasts, T lymphocytes, B lymphocytes, and natural killer cells) and kidney-specific cell types (proximal tubule (PT), distal convolved tubule, loop of Henle, two cell types forming the collecting ducts, and endothelial cells). Of these, PT is the dominant cell type in kidney, and the proportion of PT cells is known to decrease with CKD progression. Distal convolved tubule cells (DCT) are known to be the second most numerous cell type in kidney, with an expected proportion of about 10-20%. The proportion of DCT cells show a consistent increase with CKD disease progression. In addition, immune cells (particularly macrophages, but also fibroblasts, B lymphocytes, and T-lymphocytes) are known to play a central role in the pathogenesis of CKD, consistent with clinical and histological observations indicating tissue inflammation is a consistent feature of kidney fibrosis. Thus, determination of the cell type proportion of kidney cells by the methods described herein can be used to determine the presence of kidney diseases, such as CDK, and track the development and/or treatment thereof.

The methods described herein can also be used, for example, to detect the presence and extent of activated or differentiated cells within a population of cells. For example, within any population of cells, a certain percentage of the cells can be activated and another percentage of cells can be inactive. Likewise, within any population of cells, a certain percentage of the cells can be differentiated and another percentage of cells can be undifferentiated. Such stages of cells (activated versus inactive and/or differentiated versus undifferentiated) can be used in some instances to track the development of specific cells from, for example, progenitor cells to mature differentiated cells, or to track the change of normal cells to diseased cells.

In some embodiments, the methods described herein are computer-implemented. The methods may be implemented in software, hardware, firmware, or any combination thereof. In some embodiments, the methods are implemented in one or more computer programs executing on a programmable computer system including at least one processor, a storage medium readable by the processor (including, e.g., volatile and non-volatile memory and/or storage elements), and input and output devices. The computer system may comprise one or more physical machines or virtual machines running on one or more physical machines. In addition, the computer system may comprise a cluster of computers or numerous distributed computers that are connected by the Internet or other network.

Each computer program can be a set of instructions or program code in a code module resident in the random access memory of the computer system. Until required by the computer system, the set of instructions may be stored in another computer memory (e.g., in a hard disk drive, or in a removable memory such as an optical disk, external hard drive, memory card, or flash drive) or stored on another computer system and downloaded via the Internet or other network. Each computer program can be implemented in a variety of computer programming languages including, by way of example, Python.

The disclosed methods (including computer-implemented methods), computer programs, computer systems, and apparatus for deconvolving bulk RNA-sequencing data each recite, as a whole, an abundance of steps and elements well beyond an abstract idea. As an initial matter, the methods, programs, systems, and apparatus each teach a specific rules-based approach for automating the task of deconvolving bulk RNA-sequencing data. The methods, programs, systems, and apparatus each teach an ordered combination, with specific requirements defined by individual steps and elements. The specific, disclosed steps and elements of these rules are not widely prevalent and their combination is not a well understood, routine, conventional activity. Rather, the specific, disclosed steps and elements of these rules allow for the improvement realized by the disclosed methods, programs, systems, and apparatus.

Further, one focus of the disclosed methods, programs, systems, and apparatus is on the specific asserted improvement in computer capabilities; they improve the functioning of a computer itself. The improvements to a computer relevant to this disclosure include software improvements to logical structures and processes. Much of the advancement made in computer technology consists of improvements to software that, by their very nature, may not be defined by particular physical features but rather by logical structures and methods. The specific steps and elements of the disclosed methods, programs, systems, and apparatus constitute a specific type of data structure designed to improve the way a computer stores and retrieves data in memory. The disclosed methods, programs, systems, and apparatus are directed to improving the functioning of a computer and improving the technological task of deconvolving bulk RNA-sequencing data. It is the incorporation of the disclosed steps and elements, not the use of the computer, that has improved the existing technological task. Improvements in computer-related technology are not limited to improvements in the operation of a computer or a computer network per se, but may also comprise a set of “rules” (basically mathematical relationships) that improve computer-related technology.

Still further, the disclosed methods, programs, systems, and apparatus enable a computing device to do things it could not do before, such as deconvolve bulk RNA-sequencing data with higher accuracy and detect cell types at proportions less than about 0.5%. The disclosed methods, programs, systems, and apparatus provide a solution that is necessarily rooted in computer technology in order to overcome a problem specifically arising in the realm of deconvolving bulk RNA-sequencing data. As explained herein, the disclosed methods, programs, systems, and apparatus teach a specific approach for overcoming the computational limitations of the existing methods, programs, systems, and apparatus used to deconvolve bulk RNA-sequencing data. The disclosed methods, programs, systems, and apparatus overcome the deficits of the existing methods, programs, systems, and apparatus by at least accurately and efficiently deconvolving bulk RNA-sequencing data in a specific, novel, and non-obvious way.

The present disclosure also provides computer readable medium storing processor-executable instructions adapted to cause one or more computing devices to deconvolve bulk RNA-sequencing data. In some embodiments, the computer readable medium storing processor-executable instructions is adapted to cause one or more computing devices to deconvolve bulk RNA-sequencing data by any one or more of the following steps: i) selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) computing cell type-specific weights for each selected gene within the subset of most variably expressed genes within the normalized matrix of counts-based sequencing data and using cell type annotation; iii) fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; iv) fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; v) defining a loss function between the bulk distribution and the mixed single-cell distribution; and vi) applying the loss function to estimate cell type proportions in the bulk RNA-sequencing data; thereby inferring the percentage of cell types in the bulk RNA-sequencing data. Each of these embodiments can be carried out using any of the embodiments of the methods disclosed herein.

In some embodiments, the computer readable medium storing processor-executable instructions is adapted to cause one or more computing devices to deconvolve bulk RNA-sequencing data by any one or more of the following steps: i) obtaining input from three sources (bulk or spatial RNA-seq data, single cell RNA-seq data, and cell type annotations) and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the data per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities and using the model to estimate cell type proportions in the bulk RNA-sequencing data; thereby inferring the percentage of cell types in the bulk RNA-sequencing data. Each of these embodiments can be carried out using any of the embodiments of the methods disclosed herein.

In some embodiments, the methods comprise the first step. In some embodiments, the methods comprise the first step and one or more of the second, third, fourth, fifth, and sixth steps, or any combination of these additional steps. In some embodiments, the methods comprise the second step. In some embodiments, the methods comprise the second step and one or more of the first, third, fourth, fifth, and sixth steps, or any combination of these additional steps. In some embodiments, the methods comprise the third step. In some embodiments, the methods comprise the third step and one or more of the first, second, fourth, fifth, and sixth steps, or any combination of these additional steps.

In some embodiments, the methods comprise the fourth step. In some embodiments, the methods comprise the fourth step and one or more of the first, second, third, fifth, and sixth steps, or any combination of these additional steps. In some embodiments, the methods comprise the fifth step. In some embodiments, the methods comprise the fifth step and one or more of the first, second, third, fourth, and sixth steps, or any combination of these additional steps. In some embodiments, the methods comprise the sixth step. In some embodiments, the methods comprise the sixth step and one or more of the first, second, third, fourth, and fifth steps, or any combination of these additional steps.

In some embodiments, the computer readable medium storing processor-executable instructions is adapted to cause one or more computing devices to deconvolve bulk RNA-sequencing data by: i) computing the cross cell type specificity of genes; ii) estimating cross-sample gene variability from compound data or single cell samples, depending on multi-sample availability; iii) estimating gene-wise scaling factors using both compound data and single cell data; and iv) building a weighted and regularized regression model using all of the known quantities and using the model to estimate cell type proportions in the bulk RNA-sequencing data; thereby inferring the percentage of cell types in the bulk RNA-sequencing data. In some embodiments, computing the cross cell type specificity of genes is carried out based upon estimated mean and dispersion parameters of the expression per gene per cell type from a subset of the most variably expressed genes selected from a matrix of counts-based single cell sequencing data (obtained from three sources: i) bulk or spatial RNA-seq data, ii) single cell RNA-seq data, and iii) cell type annotations) wherein the matrix of counts-based sequencing data comprises counts-based sequencing counts against each gene within a plurality of genes for a fixed number of cells. Each of these embodiments can be carried out using any of the embodiments of the methods disclosed herein.

In some embodiments, the methods comprise the first step. In some embodiments, the methods comprise the first step and one or more of the second, third, and fourth steps, or any combination of these additional steps. In some embodiments, the methods comprise the second step. In some embodiments, the methods comprise the second step and one or more of the first, third, and fourth steps, or any combination of these additional steps. In some embodiments, the methods comprise the third step. In some embodiments, the methods comprise the third step and one or more of the first, second, and fourth steps, or any combination of these additional steps. In some embodiments, the methods comprise the fourth step. In some embodiments, the methods comprise the fourth step and one or more of the first, second, and third steps, or any combination of these additional steps.

The present disclosure also provides systems comprising: one or more processors; and a memory having processor executable instructions that, when executed by the one or more processors, cause the apparatus to deconvolve bulk RNA-sequencing data by any of the methods described herein. In some embodiments, the method comprises any one or more of the following steps: i) selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) computing cell type-specific weights for each selected gene within the subset of most variably expressed genes within the normalized matrix of counts-based sequencing data and using cell type annotation; iii) fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; iv) fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; v) defining a loss function between the bulk distribution and the mixed single-cell distribution; and vi) applying the loss function to estimate cell type proportions in the bulk RNA-sequencing data; thereby inferring the percentage of cell types in the bulk RNA-sequencing data.

In some embodiments, the method comprises any one or more of the following steps: i) obtaining input from three sources (bulk or spatial RNA-seq data, single cell RNA-seq data, and cell type annotations) and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the data per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities and using the model to estimate cell type proportions in the bulk RNA-sequencing data; thereby inferring the percentage of cell types in the bulk RNA-sequencing data. The deconvolution methods can be carried out using all or a subset of the embodiments of the methods disclosed herein with or without using the exact method in each embodiments described herein.

In some embodiments, the methods comprise the first step. In some embodiments, the methods comprise the first step and one or more of the second, third, fourth, fifth, and sixth steps, or any combination of these additional steps. In some embodiments, the methods comprise the second step. In some embodiments, the methods comprise the second step and one or more of the first, third, fourth, fifth, and sixth steps, or any combination of these additional steps. In some embodiments, the methods comprise the third step. In some embodiments, the methods comprise the third step and one or more of the first, second, fourth, fifth, and sixth steps, or any combination of these additional steps. In some embodiments, the methods comprise the fourth step. In some embodiments, the methods comprise the fourth step and one or more of the first, second, third, fifth, and sixth steps, or any combination of these additional steps. In some embodiments, the methods comprise the fifth step. In some embodiments, the methods comprise the fifth step and one or more of the first, second, third, fourth, and sixth steps, or any combination of these additional steps. In some embodiments, the methods comprise the sixth step. In some embodiments, the methods comprise the sixth step and one or more of the first, second, third, fourth, and fifth steps, or any combination of these additional steps.

In some embodiments, the method comprises any one or more of the following steps: i) computing the cross cell type specificity of genes; ii) estimating cross-sample gene variability from compound data or single cell samples, depending on multi-sample availability; iii) estimating gene-wise scaling factors using both compound data and single cell data; and iv) building a weighted and regularized regression model using all of the known quantities and using the model to estimate cell type proportions in the bulk RNA-sequencing data; thereby inferring the percentage of cell types in the bulk RNA-sequencing data. In some embodiments, computing the cross cell type specificity of genes is carried out based upon estimated mean and dispersion parameters of the expression per gene per cell type from a subset of the most variably expressed genes selected from a matrix of counts-based single cell sequencing data (obtained from three sources: i) bulk or spatial RNA-seq data, ii) single cell RNA-seq data, and iii) cell type annotations) wherein the matrix of counts-based sequencing data comprises counts-based sequencing counts against each gene within a plurality of genes for a fixed number of cells. The deconvolution methods can be carried out using all or a subset of the embodiments of the methods disclosed herein with or without using the exact method in each embodiments described herein.

In some embodiments, the methods comprise the first step. In some embodiments, the methods comprise the first step and one or more of the second, third, and fourth steps, or any combination of these additional steps. In some embodiments, the methods comprise the second step. In some embodiments, the methods comprise the second step and one or more of the first, third, and fourth steps, or any combination of these additional steps. In some embodiments, the methods comprise the third step. In some embodiments, the methods comprise the third step and one or more of the first, second, and fourth steps, or any combination of these additional steps. In some embodiments, the methods comprise the fourth step. In some embodiments, the methods comprise the fourth step and one or more of the first, second, and third steps, or any combination of these additional steps. The following representative embodiments are presented:

Embodiment 1. A method for deconvolving bulk RNA-sequencing data, the method comprising any one or more of the following steps: selecting a subset of the most variably expressed genes from a normalized matrix of counts-based sequencing data, wherein the matrix of counts-based sequencing data comprises counts-based sequencing counts against each gene within a plurality of genes for a fixed number of cells; computing cell type-specific weights for each selected gene within the subset of most variably expressed genes within the normalized matrix of counts-based sequencing data and using cell type annotation; fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; defining a loss function between the bulk distribution and the mixed single-cell distribution; and applying the loss function to estimate cell type proportions in the bulk RNA-sequencing data; thereby inferring the percentage of cell types in the bulk RNA-sequencing data.

Embodiment 2. The method according to embodiment 1, wherein the counts-based sequencing data is single-cell RNA-sequencing data; wherein the counts-based sequencing counts is single-cell RNA-sequencing counts; and wherein the counts-based sequencing data matrix is single-cell RNA-sequencing data matrix.

Embodiment 3. The method according to embodiment 1, wherein the counts-based sequencing data is ATAC-seq data; wherein the counts-based sequencing counts is ATAC-seq counts; and wherein the counts-based sequencing data matrix is ATAC-seq data matrix.

Embodiment 4. The method according to any one of embodiments 1 to 3, wherein the cross-sample distribution for each cell type and for each of the subset of the most variably expressed genes from the counts-based sequencing data matrix is a cross-sample Gaussian distribution.

Embodiment 5. The method according to any one of embodiments 1 to 4, wherein the bulk distribution for each subset of the most variably expressed genes from a normalized bulk matrix and a subset of the most variably expressed genes is a bulk Gaussian distribution.

Embodiment 6. The method according to any one of embodiments 1 to 5, further comprising creating the matrix of counts-based sequencing counts against each gene within the plurality of genes for a fixed number of cells and normalizing the matrix.

Embodiment 7. The method according to any one of embodiments 1 to 6, further comprising creating the bulk matrix of bulk RNA-sequencing counts and normalizing the bulk matrix.

Embodiment 8. The method according to any one of embodiments 1 to 7, wherein the step of selecting comprises calculating a standard deviation for each gene within the plurality of genes, determining a threshold standard deviation number, and selecting the subset of most variably expressed genes having a standard deviation above that threshold number.

Embodiment 9. The method according to any one of embodiments 1 to 8, wherein the step of computing cell-type specific weights comprises comparing the total mean variance to the within-cell type mean variance for each of a fixed number of cells.

Embodiment 10. The method according to any one of embodiments 1 to 9, wherein the step of fitting comprises using the whole distribution when estimating the mixing proportion.

Embodiment 11. The method according to embodiment 10, wherein the step of fitting further comprises obtaining the distribution by fitting the normalized count to the distribution and estimating the variance and mean for each gene.

Embodiment 12. The method according to embodiment 11, wherein the distribution is a Gaussian distribution.

Embodiment 13. The method according to any one of embodiments 1 to 12, wherein the step of defining a loss function comprises applying Kullback-Leibler divergence.

Embodiment 14. The method according to any one of embodiments 1 to 13, wherein the step of applying the loss function comprises adopting gradient descent.

Embodiment 15. The method according to any one of embodiments 1 to 14, wherein the plurality of genes from the normalized matrix of counts-based sequencing data comprises at least about 20,000 genes.

Embodiment 16. The method according to any one of embodiments 1 to 15, wherein the selected subset of the most variably expressed genes comprises from about 1,000 to about 5,000 genes.

Embodiment 17. The method according to embodiment 16, wherein the selected subset of the most variably expressed genes comprises about 2500 genes.

Embodiment 18. The method according to any one of embodiments 1 to 17, further comprising identifying the proportion of RNA from each cell type from which the bulk RNA-sequencing data were obtained.

Embodiment 19. The method according to any one of embodiments 1 to 18, further comprising identifying the proportion of each cell type from which the bulk RNA-sequencing data were obtained.

Embodiment 20. The method according to any one of embodiments 1 to 19, wherein the cells from which the bulk RNA-sequencing data were obtained comprise tumor cells, and the method further comprises identifying the proportion of immune cells among the tumor cells.

Embodiment 21. The method according to embodiment 20, wherein the immune cells comprise tumor infiltrating lymphocytes.

Embodiment 22. The method according to embodiment 20 or embodiment 21, wherein the immune cells comprise CD8-positive T lymphocytes.

Embodiment 23. The method according to embodiment 20, wherein the immune cells comprise CD8-positive T lymphocytes and dendritic cells.

Embodiment 24. The method according to any one of embodiments 20 to 23, further comprising characterizing the tumor from which the tumor cells were obtained as a hot tumor or a cold tumor.

Embodiment 25. The method according to embodiment 24, wherein the tumor is characterized as a hot tumor and wherein the tumor is present in a subject, and the method further comprises determining whether the subject has less than, equal to, or greater than a threshold level of infiltrating immune cells.

Embodiment 26. The method according to embodiment 25, wherein the immune cells comprise CD8-positive T lymphocytes.

Embodiment 27. The method according to embodiment 25, wherein the immune cells comprise CD8-positive T lymphocytes and dendritic cells.

Embodiment 28. The method according to any one of embodiments 25 to 27, wherein the subject has greater than a threshold level of infiltrating immune cells, and the method further comprises identifying the subject as a candidate for immunotherapy.

Embodiment 29. The method according to embodiment 28, wherein the immunotherapy comprises adoptive cell therapy.

Embodiment 30. The method according to embodiment 29, wherein the adoptive cell therapy comprises chimeric antigen receptor T-cell (CAR T-cell) therapy.

Embodiment 31. The method according to embodiment 28, wherein the immunotherapy comprises an immune checkpoint inhibition therapy.

Embodiment 32. The method according to embodiment 31, wherein the immune checkpoint inhibition therapy comprises an antibody that blocks cytotoxic T-lymphocyte-associated antigen-4 (CTLA-4).

Embodiment 33. The method according to embodiment 31 or embodiment 32, wherein the immune checkpoint inhibition therapy comprises an antibody that blocks programmed cell death protein 1 (PD-1).

Embodiment 34. The method according to any one of embodiments 31 to 33, wherein the immune checkpoint inhibition therapy comprises an antibody that blocks programmed cell death ligand 1 (PD-L1).

Embodiment 35. The method according to any one of embodiments 31 to 34, wherein the immune checkpoint inhibition therapy comprises an antibody that blocks lymphocyte-associated gene 3 (LAG3).

Embodiment 36. The method according to any one of embodiments 1 to 19, wherein the cells from which the bulk RNA-sequencing data were obtained comprise tumor microenvironment cells, and the method further comprises identifying the proportion of tumor cells among the tumor microenvironment cells.

Embodiment 37. The method according to any one of embodiments 1 to 19, wherein the cells from which the bulk RNA-sequencing data were obtained comprise tumor microenvironment cells, and the method further comprises identifying the proportion of immune cells among the tumor microenvironment cells.

Embodiment 38. The method according to any one of embodiments 1 to 19, wherein the cells from which the bulk RNA-sequencing data were obtained comprise tumor microenvironment cells, and the method further comprises identifying the proportion of cancer-associated fibroblasts among the tumor microenvironment cells.

Embodiment 39. The method according to any one of embodiments 1 to 19, wherein the cells from which the bulk RNA-sequencing data were obtained comprise tumor microenvironment cells, and the method further comprises identifying the proportion of cancer-associated adipocytes among the tumor microenvironment cells.

Embodiment 40. The method according to any one of embodiments 1 to 19, wherein the cells from which the bulk RNA-sequencing data were obtained comprise tumor microenvironment cells, and the method further comprises identifying the proportion of lymphatic endothelial cells among the tumor microenvironment cells.

Embodiment 41. The method according to any one of embodiments 1 to 19, wherein the cells from which the bulk RNA-sequencing data were obtained comprise tumor microenvironment cells, and the method further comprises identifying the proportion of blood vessel endothelial cells among the tumor microenvironment cells.

Embodiment 42. A computer readable medium storing processor-executable instructions adapted to cause one or more computing devices to deconvolve bulk RNA-sequencing data by a method comprising any one or more of the following steps: selecting a subset of the most variably expressed genes from a normalized matrix of counts-based sequencing data, wherein the matrix of counts-based sequencing data comprises counts-based sequencing counts against each gene within a plurality of genes for a fixed number of cells; computing cell type-specific weights for each selected gene within the subset of most variably expressed genes within the normalized matrix of counts-based sequencing data and using cell type annotation; fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; defining a loss function between the bulk distribution and the mixed single-cell distribution; and applying the loss function to estimate cell type proportions in the bulk RNA-sequencing data; thereby inferring the percentage of cell types in the bulk RNA-sequencing data.

Embodiment 43. The computer readable medium according to embodiment 42, wherein the counts-based sequencing data is single-cell RNA-sequencing data; wherein the counts-based sequencing counts is single-cell RNA-sequencing counts; and wherein the counts-based sequencing data matrix is single-cell RNA-sequencing data matrix.

Embodiment 44. The computer readable medium according to embodiment 42, wherein the counts-based sequencing data is ATAC-seq data; wherein the counts-based sequencing counts is ATAC-seq counts; and wherein the counts-based sequencing data matrix is ATAC-seq data matrix.

Embodiment 45. The computer readable medium according to any one of embodiments 42 to 44, wherein the cross-sample distribution for each cell type and for each of the subset of the most variably expressed genes from the counts-based sequencing data matrix is a cross-sample Gaussian distribution.

Embodiment 46. The computer readable medium according to any one of embodiments 42 to 45, wherein the bulk distribution for each subset of the most variably expressed genes from a normalized bulk matrix and a subset of the most variably expressed genes is a bulk Gaussian distribution.

Embodiment 47. The computer readable medium according to any one of embodiments 42 to 46, wherein the step of selecting comprises calculating a standard deviation for each gene within the plurality of genes, determining a threshold standard deviation number, and selecting the subset of most variably expressed genes having a standard deviation above that threshold number.

Embodiment 48. The computer readable medium according to any one of embodiments 42 to 47, wherein the step of computing cell-type specific weights comprises comparing the total mean variance to the within-cell type mean variance for each of a fixed number of cells.

Embodiment 49. The computer readable medium according to any one of embodiments 42 to 48, wherein the step of fitting comprises using the whole distribution when estimating the mixing proportion.

Embodiment 50. The computer readable medium according to embodiment 49, wherein the step of fitting further comprises obtaining the distribution by fitting the normalized count to the distribution and estimating the variance and mean for each gene.

Embodiment 51. The computer readable medium according to embodiment 50, wherein the distribution is a Gaussian distribution.

Embodiment 52. The computer readable medium according to any one of embodiments 42 to 51, wherein the step of defining a loss function comprises applying Kullback-Leibler divergence.

Embodiment 53. The computer readable medium according to any one of embodiments 42 to 52, wherein the step of applying the loss function comprises adopting gradient descent.

Embodiment 54. The computer readable medium according to any one of embodiments 42 to 53, wherein the plurality of genes from the normalized matrix of counts-based sequencing data comprises at least about 20,000 genes.

Embodiment 55. The computer readable medium according to any one of embodiments 42 to 54, wherein the selected subset of the most variably expressed genes comprises from about 1,000 to about 5,000 genes.

Embodiment 56. The computer readable medium according to embodiment 55, wherein the selected subset of the most variably expressed genes comprises about 2500 genes.

Embodiment 57. The computer readable medium according to any one of embodiments 42 to 56, wherein the method further comprises identifying the proportion of RNA from each cell type from which the bulk RNA-sequencing data were obtained.

Embodiment 58. The computer readable medium according to any one of embodiments 42 to 57, wherein the method further comprises identifying the proportion of each cell type from which the bulk RNA-sequencing data were obtained.

Embodiment 59. A system comprising: one or more processors; and a memory having processor executable instructions that, when executed by the one or more processors, cause the apparatus to deconvolve bulk RNA-sequencing data by a method comprising any one or more of the following steps: selecting a subset of the most variably expressed genes from a normalized matrix of counts-based sequencing data, wherein the matrix of counts-based sequencing data comprises counts-based sequencing counts against each gene within a plurality of genes for a fixed number of cells; computing cell type-specific weights for each selected gene within the subset of most variably expressed genes within the normalized matrix of counts-based sequencing data and using cell type annotation; fitting a cross-sample distribution for each cell type and for each of the subset of the most variably expressed genes from the counts-based sequencing data matrix, the subset of the most variably expressed genes, and the cell type annotation, and defining a mixed single-cell distribution with proportion parameters; fitting a bulk distribution for each subset of the most variably expressed genes from a normalized bulk matrix and a subset of the most variably expressed genes, and defining a bulk distribution, wherein the bulk matrix comprises bulk RNA-sequencing counts against each gene within a plurality of genes for a fixed number of cells; defining a loss function between the bulk distribution and the mixed single-cell distribution; and applying the loss function to estimate cell type proportions in the bulk RNA-sequencing data; thereby inferring the percentage of cell types in the bulk RNA-sequencing data.

Embodiment 60. The system according to embodiment 59, wherein the counts-based sequencing data is single-cell RNA-sequencing data; wherein the counts-based sequencing counts is single-cell RNA-sequencing counts; and wherein the counts-based sequencing data matrix is single-cell RNA-sequencing data matrix.

Embodiment 61. The system according to embodiment 59, wherein the counts-based sequencing data is ATAC-seq data; wherein the counts-based sequencing counts is ATAC-seq counts; and wherein the counts-based sequencing data matrix is ATAC-seq data matrix.

Embodiment 62. The system according to any one of embodiments 59 to 61, wherein the cross-sample distribution for each cell type and for each of the subset of the most variably expressed genes from the counts-based sequencing data matrix is a cross-sample Gaussian distribution.

Embodiment 63. The system according to any one of embodiments 59 to 62, wherein the bulk distribution for each subset of the most variably expressed genes from a normalized bulk matrix and a subset of the most variably expressed genes is a bulk Gaussian distribution.

Embodiment 64. The system according to any one of embodiments 59 to 63, wherein the step of selecting comprises calculating a standard deviation for each gene within the plurality of genes, determining a threshold standard deviation number, and selecting the subset of most variably expressed genes having a standard deviation above that threshold number.

Embodiment 65. The system according to any one of embodiments 59 to 64, wherein the step of computing cell-type specific weights comprises comparing the total mean variance to the within-cell type mean variance for each of a fixed number of cells.

Embodiment 66. The system according to any one of embodiments 59 to 65, wherein the step of fitting comprises using the whole distribution when estimating the mixing proportion.

Embodiment 67. The system according to embodiment 66, wherein the step of fitting further comprises obtaining the distribution by fitting the normalized count to the distribution and estimating the variance and mean for each gene.

Embodiment 68. The system according to embodiment 67, wherein the distribution is a Gaussian distribution.

Embodiment 69. The system according to any one of embodiments 59 to 68, wherein the step of defining a loss function comprises applying Kullback-Leibler divergence.

Embodiment 70. The system according to any one of embodiments 59 to 69, wherein the step of applying the loss function comprises adopting gradient descent.

Embodiment 71. The system according to any one of embodiments 59 to 70, wherein the plurality of genes from the normalized matrix of counts-based sequencing data comprises at least about 20,000 genes.

Embodiment 72. The system according to any one of embodiments 59 to 70, wherein the selected subset of the most variably expressed genes comprises from about 1,000 to about 5,000 genes.

Embodiment 73. The system according to embodiment 72, wherein the selected subset of the most variably expressed genes comprises about 2500 genes.

Embodiment 74. The system according to any one of embodiments 59 to 73, wherein the method further comprises identifying the proportion of RNA from each cell type from which the bulk RNA-sequencing data were obtained.

Embodiment 75. The system according to any one of embodiments 59 to 74, wherein the method further comprises identifying the proportion of each cell type from which the bulk RNA-sequencing data were obtained.

Embodiment 76. A method for deconvolving bulk or spatial RNA-sequencing data, the method comprising any one or more of the following steps: a) obtaining input from sources comprising bulk or spatial RNA-seq data, single cell RNA-seq data, and cell type annotations, and selecting a subset of the most variably expressed genes from a normalized matrix of counts-based sequencing data; b) estimating the mean and dispersion parameters of the expression per gene per cell type; c) computing the cross cell type specificity of each gene; d) estimating cross-sample gene variability from the bulk or spatial RNA-seq data or single cell samples depending on multi-sample availability; e) estimating gene-wise scaling factors using both the bulk or spatial RNA-seq data and single cell data; and f) building a weighted and regularized regression model using all of the known quantities and using the model to estimate cell type proportions in the bulk or spatial RNA-sequencing data; thereby inferring the percentage of cell types in the bulk or spatial RNA-sequencing data.

Embodiment 77. The method according to embodiment 76, wherein the matrix of counts-based sequencing data comprises counts-based sequencing counts against each gene within a plurality of genes for a fixed number of cells.

Embodiment 78. The method according to embodiment 76 or embodiment 77, wherein the input is a single cell UMI count matrix with cell type annotations associated with each cell.

Embodiment 79. The method according to embodiment 76 or embodiment 77, wherein the bulk data to be deconvolved is transcripts per kilobase million (TPM) or read counts.

Embodiment 80. The method according to embodiment 76 or embodiment 77, wherein the spatial data to be deconvolved is a UMI count matrix.

Embodiment 81. The method according to any one of embodiments 76 to 80, wherein genes that contain important information to differentiate cell types, excluding non-informative genes that potentially introduce noise, are selected as the subset of the most variably expressed genes from a normalized matrix of counts-based sequencing data.

Embodiment 82. The method according to embodiment 81, wherein the subset of genes is selected from the union of marker genes whose expression is enriched in each cell type in the single cell UMI count matrix.

Embodiment 83. The method according to embodiment 82, wherein a build-in tool takes as input the single cell UMI count matrix and cell type annotations.

Embodiment 84. The method according to embodiment 83, wherein for each cell type, the tool computes the fold change between the average UMI in that cell type and the average UMI in all other cell types, and ranks genes by descending fold changes.

Embodiment 85. The method according to embodiment 84, wherein about the top 200 genes from each cell type are selected.

Embodiment 86. The method according to embodiment 84, wherein selected marker genes presenting in no more than five cell types are selected.

Embodiment 87. The method according to embodiment 84, wherein selected marker genes presenting in a fixed number of cell types or a proportion of total number of cell types, whichever is smaller, are selected.

Embodiment 88. The method according to embodiment 84, wherein about 1,000 total unique genes are selected.

Embodiment 89. The method according to embodiment 81, wherein the subset of genes is selected from the union of highly variable genes that vary the most across all the cells in the single cell UMI count matrix.

Embodiment 90. The method according to embodiment 89, wherein variances for each gene after cell number balancing and VST normalization are computed.

Embodiment 91. The method according to embodiment 90, wherein genes with the highest variances are selected.

Embodiment 92. The method according to any one of embodiments 89 to 91, wherein the cell types in the single cell UMI count matrix are balanced by finding the median size of all the cell clusters, wherein cells from each cluster are sampled to make them equal to this size.

Embodiment 93. The method according to embodiment 92, wherein the variance of each gene across the cells in the balanced single cell UMI matrix is computed.

Embodiment 94. The method according to embodiment 93, wherein variances on the normalized data are computed by variance stabilization transformed (VST).

Embodiment 95. The method according to embodiment 94, wherein genes with the top 2,000 large variances are selected.

Embodiment 96. The method according to any one of embodiments 76 to 95, wherein the RNA-seq data is not normalized before estimating the mean.

Embodiment 97. The method according to embodiment 96, wherein the mean is modeled using the raw UMI counts.

Embodiment 98. The method according to embodiment 96 or embodiment 97, wherein negative binomial distributions are fit to single cells of each cell type.

Embodiment 99. The method according to embodiment 98, wherein estimations are performed for each selected gene in each cell type.

Embodiment 100. The method according to any one of embodiments 76 to 99, wherein to compute the cell type specificity of a gene, the cell type in which the gene has: i) the highest expression, or ii) highest fold change compared to others, is identified, and the specificity of this gene is defined as the mean-to-variance ratio within the cell type.

Embodiment 101. The method according to embodiment 100, wherein the estimated mean and variance parameters from the negative binomial fitting is used to compute the cell type specificity weight for each gene in the set of selected genes.

Embodiment 102. The method according to any one of embodiments 76 to 101, wherein the cross-sample gene variability is computed using the variance-to-mean ratio (VMR) computed across samples.

Embodiment 103. The method according to embodiment 102, wherein the cross-sample gene variability is computed from compound transcriptome data.

Embodiment 104. The method according to embodiment 102, wherein the compound data do not have multi-samples whereas the single cell data have multiple samples, and multiple compound samples are synthesized, each of which is an average of all cells belonging to one of the samples in the single cell reference.

Embodiment 105. The method according to embodiment 102, wherein if multi-samples are unavailable for both compound data and single cell data, the method comprises generating multiple synthetic samples for the single cell data by averaging the expression of subset of cells.

Embodiment 106. The method according to any one of embodiments 76 to 105, wherein the gene-wise scaling factors are estimated using an adaptive learning strategy, and each gene is rescaled with its respective scaling factor.

Embodiment 107. The method according to any one of embodiments 76 to 106, wherein each compound sample is independently estimated by the regression model.

Embodiment 108. A method for deconvolving bulk or spatial RNA-sequencing data, the method comprising any one or more of the following steps: a) computing the cross cell type specificity of each gene within a subset of the most variably expressed genes selected from a normalized matrix of counts-based sequencing data obtained from sources comprising bulk or spatial RNA-seq data, single cell RNA-seq data, and cell type annotations; b) estimating cross-sample gene variability from the bulk or spatial RNA-seq data or single cell samples depending on multi-sample availability; c) estimating gene-wise scaling factors using both the bulk or spatial RNA-seq data and single cell data; and d) building a weighted and regularized regression model using all of the known quantities and using the model to estimate cell type proportions in the bulk or spatial RNA-sequencing data; thereby inferring the percentage of cell types in the bulk or spatial RNA-sequencing data.

Embodiment 109. A computer readable medium storing processor-executable instructions adapted to cause one or more computing devices to deconvolve bulk or spatial RNA-sequencing data by a method comprising any one or more of the following steps: i) obtaining input from sources comprising bulk or spatial RNA-seq data, single cell RNA-seq data, and cell type annotations, and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the expression per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities, and using the model to estimate cell type proportions in the bulk or spatial RNA-sequencing data; thereby inferring the percentage of cell types in the bulk or spatial RNA-sequencing data.

Embodiment 110. A computer readable medium storing processor-executable instructions adapted to cause one or more computing devices to deconvolve bulk or spatial RNA-sequencing data, by a method comprising any one or more of the following steps: i) computing the cross cell type specificity of genes within a subset of the most variably expressed genes selected from a normalized matrix of counts-based sequencing data obtained from sources comprising bulk or spatial RNA-seq data, single cell RNA-seq data, and cell type annotations; ii) estimating cross-sample gene variability from compound data or single cell samples, depending on multi-sample availability; iii) estimating gene-wise scaling factors using both compound data and single cell data; and iv) building a weighted and regularized regression model using all of the known quantities, and using the model to estimate cell type proportions in the bulk or spatial RNA-sequencing data; thereby inferring the percentage of cell types in the bulk or spatial RNA-sequencing data.

Embodiment 111. A system comprising: one or more processors; and a memory having processor executable instructions that, when executed by the one or more processors, cause the apparatus to deconvolve bulk or spatial RNA-sequencing data by a method comprising any one or more of the following steps: i) obtaining input from sources comprising bulk or spatial RNA-seq data, single cell RNA-seq data, and cell type annotations, and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the data per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities and using the model to estimate cell type proportions in the bulk or spatial RNA-sequencing data; thereby inferring the percentage of cell types in the bulk or spatial RNA-sequencing data.

Embodiment 112. A system comprising: one or more processors; and a memory having processor executable instructions that, when executed by the one or more processors, cause the apparatus to deconvolve bulk or spatial RNA-sequencing data, by a method comprising any one or more of the following steps: i) computing the cross cell type specificity of genes within a subset of the most variably expressed genes selected from a normalized matrix of counts-based sequencing data obtained from sources comprising bulk or spatial RNA-seq data, single cell RNA-seq data, and cell type annotations; ii) estimating cross-sample gene variability from compound data or single cell samples, depending on multi-sample availability; iii) estimating gene-wise scaling factors using both compound data and single cell data; and iv) building a weighted and regularized regression model using all of the known quantities and using the model to estimate cell type proportions in the bulk or spatial RNA-sequencing data; thereby inferring the percentage of cell types in the bulk or spatial RNA-sequencing data.

In order that the subject matter disclosed herein may be more efficiently understood, examples are provided below. It should be understood that these examples are for illustrative purposes only and are not to be construed as limiting the claimed subject matter in any manner.

### Example 1: Deconvolving Bulk RNA-Sequencing Data for Immune Cells

The following hypothetical example is included to more clearly demonstrate the overall nature of the disclosure. The example is exemplary, not restrictive, of the disclosure.

For a single-cell matrix that contains three cell types: macrophage, T cells, and B cells, there are three genes (Spp1, Trem2, and Serpine2) measured by RNA-sequencing.

### Single Cell Data Input: Raw UMI Counts Data

| Gene | Macrophage | | | T Cells | | | B Cells | | |
|---|---|---|---|---|---|---|---|---|---|
| Spp1 | 4 | 60 | 8 | 4 | 53 | 48 | 0 | 0 | 10 |
| Trem2 | 23 | 5950 | 21 | 0 | 3 | 2 | 15 | 22 | 120 |
| Serpine2 | 0 | 0 | 1 | 0 | 1 | 0 | 0 | 0 | 0 |

Normalize to 1e+6 Total UMI Counts Per Column:

| Gene | Macrophage | | | T Cells | | | B Cells | | |
|---|---|---|---|---|---|---|---|---|---|
| Spp1 | 4 | 6 | 8 | 40 | 53 | 48 | 0 | 0 | 1 |
| Trem2 | 232 | 595 | 213 | 0 | 3 | 2 | 15 | 22 | 12 |
| Serpine2 | 0 | 0 | 1 | 0 | 1 | 0 | 0 | 0 | 0 |

### Add 1 to Every Number:

| Gene | Macrophage | | | T Cells | | | B Cells | | |
|---|---|---|---|---|---|---|---|---|---|
| Spp1 | 5 | 7 | 9 | 41 | 54 | 49 | 0 | 0 | 2 |
| Trem2 | 233 | 596 | 214 | 1 | 4 | 3 | 16 | 23 | 13 |
| Serpine2 | 1 | 1 | 2 | 1 | 2 | 1 | 1 | 1 | 1 |

### Calculate Natural Log Transform:

| Gene | Macrophage | | | T Cells | | | B Cells | | |
|---|---|---|---|---|---|---|---|---|---|
| Spp1 | 1.6 | 1.9 | 2.2 | 3.7 | 3.99 | 3.89 | 0 | 0 | 0.69 |
| Trem2 | 5.45 | 6.39 | 5.37 | 0 | 1.39 | 1.1 | 2.77 | 3.13 | 2.56 |
| Serpine2 | 0 | 0 | 0.69 | 0 | 0.69 | 0 | 0 | 0 | 0 |

Applying Step 1 of the disclosed method, the top 2,500 highly variable genes are selected. In this case, the gene Serpine2 does not have much variance. The other two genes, Spp1 and Trem2, have greater variability and, therefore, are included among the selected top 2,500 genes for further analysis.

In step 2, the cell-type specific weights are calculated.

| Gene | Macrophage | | | T Cells | | | B Cells | | |
|---|---|---|---|---|---|---|---|---|---|
| Spp1 | 1.6 | 1.9 | 2.2 | 3.7 | 3.99 | 3.89 | 0 | 0 | 0.69 |
| Trem2 | 5.45 | 6.39 | 5.37 | 0 | 1.39 | 1.1 | 2.77 | 3.13 | 2.56 |

### The Calculation for the Spp1 Gene Follows:

$${W}_{Spp 1 , Macrophage} = \frac{{σ}_{Spp 1}^{2} / ( N - 1 )}{{σ}_{Spp 1 , Macrophage}^{2} / ( \begin{matrix}{n}_{Macrophage} & 1\end{matrix} )} - \frac{2.54 / ( 9 - 1 )}{0.09 / ( 3 - 1 )} = 7.05 .$$

In the example, 2.54 is the unbiased variance of (1.6, 1.9, 2.2, 3.7, 3.99, 3.89, 0, 0, 0.69); N is the total number of cells (3+3+3=9); 0.09 is the unbiased variance of (1.6, 1.9, 2.2); and n is the number of macrophage cells (3). Applying similar calculations, the cell type specific weights are:

| Gene | Macrophage | T Cells | B Cells |
|---|---|---|---|
| Spp1 | 7.05 | 29.3 | 4 |
| Trem2 | 3.73 | 2.23 | 14.45 |

Applying Step 3 of the disclosed methods, the single cells are pooled per cell type, then normalized to the total UMI per cell-type column=1e+6, then 1 is added, then a natural log transformation is performed. The results are reflected in the tables below for each of three samples.

### Sample 1:

| Gene | Macrophage | | | T cells | | | B cells | | |
|---|---|---|---|---|---|---|---|---|---|
| Spp1 | 5 | 7 | 9 | 41 | 54 | 46 | 0 | 1 | 0 |
| Trem2 | 231 | 487 | 209 | 1 | 1 | 2 | 13 | 21 | 12 |

### Pool (Sum) Cells Per Cell Type to Produce

| Gene | Macrophage | T cells | B cells |
|---|---|---|---|
| Spp1 | 21 | 141 | 1 |
| Trem2 | 927 | 4 | 46 |

### Normalization then Log(Data+1) to Produce

| Gene | Macrophage | T cells | B cells |
|---|---|---|---|
| Spp1 | 3.09 | 4.95 | 1 |
| Trem2 | 6.83 | 1.79 | 3.85 |

### Sample 2:

| Gene | Macrophage | | | T cells | | | B cells | | |
|---|---|---|---|---|---|---|---|---|---|
| Spp1 | 4 | 6 | 8 | 42 | 53 | 48 | 0 | 0 | 0 |
| Trem2 | 232 | 595 | 213 | 0 | 3 | 2 | 15 | 22 | 12 |

### Pool (Sum) Cells Per Cell Type to Produce

| Gene | Macrophage | T cells | B cells |
|---|---|---|---|
| Spp1 | 18 | 143 | 0 |
| Trem2 | 1040 | 5 | 49 |

### Normalization then Log(Data+1) to Produce

| Gene | Macrophage | T cells | B cells |
|---|---|---|---|
| Spp1 | 3.02 | 4.98 | 0 |
| Trem2 | 6.98 | 2.25 | 3.89 |

### Sample 3:

| Gene | Macrophage | | | T cells | | | B cells | | |
|---|---|---|---|---|---|---|---|---|---|
| Spp1 | 3 | 8 | 6 | 40 | 58 | 41 | 1 | 0 | 1 |
| Trem2 | 212 | 582 | 205 | 1 | 3 | 1 | 12 | 28 | 14 |

### Pool (Sum) Cells Per Cell Type to Produce

| Gene | Macrophage | T cells | B cells |
|---|---|---|---|
| Spp1 | 17 | 139 | 2 |
| Trem2 | 999 | 5 | 54 |

### Normalization then Log(Data+1) to Produce

Normalization then log(data + 1) to produce:

| Gene | Macrophage | T cells | B cells |
|---|---|---|---|
| Spp1 | 3.01 | 4.87 | 1 |
| Trem2 | 6.92 | 2.25 | 3.92 |

Continuing with Step 3, a Gaussian distribution is fit across each of the three samples for each gene of the two genes in each of the three cell types. The results of these Gaussian distribution fits are shown in FIG. 8. These results conclude the application of Step 3.

Applying Step 4 of the disclosed methods, the multi-sample matrix is normalized and Gaussian distributions are fit for each gene. The results of these Gaussian distribution fits are shown in FIG. 9. An assumption is that the bulk RNA-seq data is independently acquired from three samples. The cell content in each sample is unknown. One goal of the disclosed methods is to learn the cell-type proportion for each sample, using the previous single cell RNA-seq data as a reference.

Applying Step 5 of the disclosed methods, the model is defined using the proportion parameters, weights, and distributions of each gene learned from the single-cell data and from the bulk data. The results of these calculations are shown in FIG. 10.

Finally, applying Step 6 of the disclosed methods, the proportion θₖ of each of the three cell types (k=1, 2, 3) is estimated in the bulk tissue by minimizing the total Dₖᵢ for each of the three samples. The output for the first sample is: θ₁ (macrophage)=0.5; 02 (T cells)=0.22; and θ₃ (B cells)=0.28. The output for the second sample is: θ₁ (macrophage)=0.42; θ₂ (T cells)=0.32; and θ₃ (B cells)=0.26. The output for the third sample is: θ₁ (macrophage)=0.23; θ₂ (T cells)=0.38; and θ₃ (B cells)=0.39. Of course, the total of the proportions of the three cell types must equal 1.

### Example 2: Method Evaluation

To evaluate the AdRoit methods, two comparisons were made. First, the results of the second embodiment of the AdRoit methods were compared to results achieved by the “MUlti-Subject Single Cell deconvolution” (MuSiC) method disclosed in the article by X. Wang et al. (supra). Second, the results of the second embodiment of the AdRoit methods were compared to results achieved by a conventional, non-negative least squares (NNLS) regression method.

### Evaluation 1: Human Islets Data

The data used for the first evaluation were taken from human islets. The islets of Langerhans are the regions of the pancreas that contain its endocrine (i.e., hormone-producing) cells. The human islets single-cell data are shown in FIG. 15A and FIG. 15B. These data were selected for the comparisons because the data include several (specifically, four) cell types from many (specifically, 18) subjects, including two major cell types (Alpha and Beta cells) and two minor cell types (PP and Delta cells). The cell fractions vary across the different subjects.

FIG. 15A is a summary of the cell composition of the 18 subjects. FIG. 15B reflects T-distributed Stochastic Neighbor Embedding (t-SNE), which is a machine learning algorithm for visualization developed by Laurens van der Maaten and Geoffrey Hinton. It is a nonlinear dimensionality reduction technique well-suited for embedding high-dimensional data for visualization in a low-dimensional space of two dimensions (such as the graph of FIG. 15B). Specifically, t-SNE models each high-dimensional object by a two-dimensional point in such a way that similar objects are modeled by nearby points and dissimilar objects are modeled by distant points with high probability. As is typical of t-SNE graphs, the data are displayed in clusters in FIG. 15B.

The bulk data corresponding to the human islet single-cell data shown in FIG. 15A and FIG. 15B were synthesized to obtain the absolute, correct, or “true” results used to evaluate the different methods. Estimation was carried out by using the single cell reference without the sample used to synthesize the bulk data. The true bulk data are reflected in FIGS. 16A, 16B, and 16C. Four, separate, statistical measurements were calculated: the mean absolute deviation (mAD), the root-mean-square deviation (RMSD), the Pearson correlation coefficient (PCC), and Spearman's rank correlation coefficient.

In statistics, the mAD is the mean of the absolute deviations of a set of data about the mean of that data. The mean absolute deviation is also called the mean deviation. Mean absolute deviation is a way to describe variation in a data set. The lower the mAD number, the less the variation in a data set (i.e., the better).

The RMSD or root-mean-square error (RMSE) (or sometimes root-mean-squared error) is a frequently used measure of the differences between values (sample or population values) predicted by a model or an estimator and the values observed. The RMSD represents the square root of the second sample moment of the differences between predicted values and observed values or the quadratic mean of these differences. These deviations are called residuals when the calculations are performed over the data sample that was used for estimation and are called errors (or prediction errors) when computed out-of-sample. The RMSD serves to aggregate the magnitudes of the errors in predictions for various times into a single measure of predictive power. RMSD is a measure of accuracy, to compare forecasting errors of different models for a particular dataset and not between datasets, as it is scale-dependent. As for mAD, the lower the RMSD number, the better.

In statistics, the PCC, also referred to as Pearson's r, the Pearson product-moment correlation coefficient (PPMCC) or the bivariate correlation, is a measure of the linear correlation between two variables X and Y. According to the Cauchy-Schwarz inequality it has a value between +1 and −1, where +1 is total positive linear correlation, 0 is no linear correlation, and −1 is total negative linear correlation. The PCC is widely used in the sciences. It was developed by Karl Pearson from a related idea introduced by Francis Galton in the 1880s and for which the mathematical formula was derived and published by Auguste Bravais in 1844.

In statistics, Spearman's rank correlation coefficient or Spearman's rho, named after Charles Spearman and often denoted by the Greek letter p (rho) or as r₅, is a nonparametric measure of rank correlation (statistical dependence between the rankings of two variables). It assesses how well the relationship between two variables can be described using a monotonic function. The Spearman correlation between two variables is equal to the Pearson correlation between the rank values of those two variables; while Pearson's correlation assesses linear relationships, Spearman's correlation assesses monotonic relationships (whether linear or not). For both Pearson's correlation and Spearman's correlation, the closer the number is to 1, the better.

FIGS. 16A, 16B, and 16C reflect a comparison among the results of the AdRoit method (FIG. 16A), the MuSiC method (FIG. 16B), and the NNLS method (FIG. 16C). Thus, three graphs are depicted. The ordinate (Y axis) for each graph is the estimated proportion of cell types provided by the respective method. The abscissa (X axis) for each graph is the true proportion of cell types (from the synthesized bulk data). The four, separate, statistical measurements (summarized above) were calculated for each of the three graphs and are tabulated in FIG. 17. For the mAD and RMSD measurements, the lower the number (i.e., the smaller the deviation) the more accurate the method. The Spearman and Pearson correlations approach 1 as the method becomes more accurate. The data support the conclusion that the AdRoit method is both highly accurate and superior to the MuSiC and NNLS methods. AdRoit achieves leading accuracy when applied to the synthetic bulk data using human islets single cell data.

### Evaluation 2: Human Trabecular Meshwork Data

The data used for the second evaluation were taken from human trabecular meshworks (TM). The TM is an area of tissue in the eye located around the base of the cornea, near the ciliary body, and is responsible for draining the aqueous humor from the eye via the anterior chamber (the chamber on the front of the eye covered by the cornea). The human TM single-cell data are shown in FIG. 18A and FIG. 18B. These data were selected for the comparisons because the data include a large number (specifically, 12) of cell types from many (specifically, eight) donors. See Patel, G. et al., “Molecular taxonomy of human ocular outflow tissues defined by single-cell transcriptomics,” Proc. Natl. Acad. Sci. 117, 12856 LP-12867 (2020). The cell types are listed in FIG. 18A. The cell fractions vary across the different donors.

FIG. 18A is a summary of the cell composition of the eight donors. FIG. 18B reflects t-SNE visualization and, as is typical of t-SNE graphs, the data are displayed in clusters in FIG. 18B.

The bulk data corresponding to the human TM single-cell data shown in FIG. 18A and FIG. 18B were synthesized to obtain the absolute, correct, or “true” results used to evaluate the different methods. Estimation was done by using the single cell reference without the donor used to synthesize the bulk data. The true bulk data are reflected in FIGS. 19A, 19B, and 19C. Again, four, separate, statistical measurements were calculated: the mAD, the RMSD, the PCC, and Spearman's rank correlation coefficient.

FIGS. 19A, 19B, and 19C reflect a comparison among the results of the AdRoit method (FIG. 19A), the MuSiC method (FIG. 19B), and the NNLS method (FIG. 19C). Thus, three graphs are depicted. The ordinate (Y axis) for each graph is the estimated proportion of cell types provided by the respective method. The abscissa (X axis) for each graph is the true proportion of cell types (from the synthesized bulk data). The four, separate, statistical measurements (summarized above) were calculated for each of the three graphs and are tabulated in FIG. 20. Overall, the AdRoit method has the closest estimation to the true cell fractions compared to the MuSiC and NNLS methods. The AdRoit method has the lowest mAD and RMSD, and the highest Pearson and Spearman correlations.

FIG. 21 shows 12 bar graphs, one for each of the cell types. Dots on the graphs denote each of the eight different donors, and bars represent the 1.5× interquartile range. For each cell type in TM, the AdRoit method had the smallest differences from the true cell fractions, and the tightest estimation across the eight different donors.

FIG. 22 reflects estimated and true data calculated using both the AdRoit method and the MuSiC method. The synthetic bulk data were simulated by using only six of the 12 cell types, then estimated with reference to the full list of all 12 cell types. The AdRoit method had fewer false positive estimates of the six cell types excluded in the simulation, and more accurate estimation of the six cell types included in the simulation.

FIG. 23 is a receiver operating characteristic (ROC) curve showing that the AdRoit method had a significantly higher area under curve (AUC) than the MuSiC method, indicating better sensitivity and specificity. A ROC curve is a graphical plot that illustrates the diagnostic ability of a binary classifier system as its discrimination threshold is varied. The diagnostic method was developed for operators of military radar receivers, which is why it is so named. The ROC curve is created by plotting the true positive rate (TPR) against the false positive rate (FPR) at various threshold settings. The TPR is also known as sensitivity. The FPR is also known as probability of false alarm and can be calculated as 1-specificity. ROC analysis provides a tool to select possibly optimal models and to discard suboptimal ones.

### Evaluation 3: Dorsal Root Ganglion Data

The data used for the third evaluation were taken from mouse dorsal root ganglion (DRG) neurons. The DRG single-cell RNA-seq data are shown in FIG. 24A and FIG. 24B. These data were selected for the comparisons because the data include many (specifically, 14) cell types, including multiple subtypes of neuronal cells, from five mice. FIG. 24A is a summary of the cell composition of the five mice and lists the cell types. FIG. 24B reflects t-SNE visualization of the data. The cell fractions vary across the different mice.

The bulk data corresponding to the DRG single-cell data shown in FIG. 24A and FIG. 24B were synthesized to obtain the absolute, correct, or “true” results used to evaluate the different methods. Estimation was done by using the single cell reference without the sample used to synthesize the bulk data. The true bulk data are reflected in FIGS. 25A, 25B, and 25C.

FIGS. 25A, 25B, and 25C reflect a comparison among the results of the AdRoit method (FIG. 25A), the MuSiC method (FIG. 25B), and the NNLS method (FIG. 25C). Thus, three graphs are depicted. The ordinate (Y axis) for each graph is the estimated proportion of cell types provided by the respective method. The abscissa (X axis) for each graph is the true proportion of cell types (from the synthesized bulk data). For each individual sample, mAD, RMSD, and Pearson and Spearman correlations were computed and compared among the three methods. The results are shown in the graphs of FIG. 26. Overall, the AdRoit method has the closest estimation to the true cell fractions compared to the MuSiC and NNLS methods. The AdRoit method has the lowest mAD and RMSD, and the highest Pearson and Spearman correlations. In addition, the AdRoit method estimation was the most stable across samples.

### Evaluation 4: Human Islets Applications

The data used for the fourth evaluation were taken from human islets (see Evaluation 1 above). The human islets single-cell data are shown in FIG. 15A and FIG. 15B. These data include four cell types: Alpha, Beta, PP, and Delta cells. FIG. 27 is a graph of cell fraction for each of the four cell types and shows that the AdRoit method estimations of cell type percentages on real human islets bulk RNA-seq data are highly reproducible for repeated samples from the same donor.

Fluorescent in situ hybridization targeting ribonucleic acid molecules (RNA FISH) is a methodology for detecting and localizing particular RNA molecules in fixed cells. This detection uses nucleic acid probes that are complementary to target RNA sequences within the cell. These probes then hybridize to their targets via standard Watson-Crick base pairing, after which they can be detected via fluorescence microscopy, either through direct conjugation of fluorescent molecules to the probe or through fluorescent signal amplification schemes. Recent advances in RNA FISH have increased the specificity and sensitivity of the method to enable the detection of individual RNA molecules, providing accurate measurements of RNA abundance and localization at the single cell or even subcellular level. Although most applications thus far have been in fixed cells, advances in probe technology have led to the ability to detect single RNA molecules in living cells. FIG. 28 shows that cell fraction percentages estimated using the Adroit method agree with the RNA FISH measurements of cell-type percentages.

Glycated hemoglobin, or HbA1c, is made when the glucose (sugar) in the human body sticks to red blood cells. Tests of HbA1c are used to monitor type 2 diabetes (T2D) patients. In such patients, the body cannot use sugar properly and the sugar tends to stick to blood cells and build up in the blood. Red blood cells are active for about two-three months, which is why HbA1c tests are taken quarterly. A high HbA1c means the patient has too much sugar in their blood and is more likely to develop diabetes complications, such as problems with their eyes and feet. In T2D patients, an ideal HbA1c level is 48 mmol/mol (6.5%) or below. FIG. 29 shows that Beta cell fraction percentages estimated using the Adroit method have a significant linear relationship with donors' HbA1C levels (including both healthy and T2D cells). FIG. 30 shows that Beta cell fraction percentages estimated using the Adroit method in T2D patients are significantly lower than in healthy subjects.

### Evaluation 5: Simulated Spatial Spots

The data used for the fifth evaluation compare the AdRoit method with stereoscope estimations. Spatial transcriptomics (ST) is a technology used to spatially resolve RNA-seq data, and thereby all mRNAs, in individual tissue sections. The ordered attachment of spatially barcoded reverse transcription oligo(dT) primers to the surface of microscope slides in an array of spots enables the encoding and maintenance of positional information throughout mRNA sample processing and subsequent sequencing. This contrasts with RNA-sequencing of single cells, or the sequencing of bulk RNA extracted from tissue volumes, where precise spatial information is lost. When a tissue cryosection is attached to a spatial transcriptomic slide the barcoded primers bind and capture adjacent mRNAs from the tissue. While the tissue section is attached to the slide, reverse transcription of captured mRNA is initiated and the resulting cDNA incorporates the spatial barcode of the primer. Following mRNA capture and reverse transcription, sequencing libraries are prepared and analyzed with Illumina dye sequencing. The spatial barcode present within each generated sequence allows the data for each individual mRNA transcript to be mapped back to its point of origin within the tissue section.

Stereoscopy (also called stereoscopics or stereo imaging) is a technique for creating or enhancing the illusion of depth in an image by means of stereopsis for binocular vision. A stereoscope is a type of image viewer that creates the illusion of a three dimensional image from two similar, two-dimensional images through the use of mirrors or lenses. Complex cellular structures are often rendered in stereopairs.

FIG. 31 compares estimations achieved by stereoscopy and the AdRoit method on simulated spatial spots that contain five different PEP cell subtypes. True mixing fractions are denoted by the vertical, red, dashed lines. Three schemes were simulated: (1) in Scheme 1, on the left of FIG. 31, fractions of the five PEP cell types were the same and equal to 0.2; (2) in Scheme 2, in the middle of FIG. 31, one PEP cell type was 0.1 and the other four were 0.225; and (3) in Scheme 3, on the right of FIG. 31, two PEP cell types were 0.1, two were 0.2, and one was 0.4. In all simulation schemes, the AdRoit estimates were more consistently centered around the true simulated fractions than were the stereoscope estimates.

FIG. 32 illustrates simulation of a very low percent of a single type of PEP cell. The percentages were 0.02, 0.04, 0.06, 0.08, and 0.1. True mixing fractions are denoted by the horizontal, red, dashed lines. The medians of the estimates achieved using the AdRoit method were close to the true fractions and closer than the estimates achieved using stereoscopy. FIG. 33 compares estimates using stereoscopy and the AdRoit method via graphs of detection rate versus simulated fraction for very low amounts of six different cell types. The AdRoit method was more sensitive in detecting low percent cells, and also more consistent across different mixtures of cell types.

### Evaluation 6: Mouse Brain Spatial Transcriptome Application

The data used for the sixth evaluation were taken from mouse brain cell types. The Allen mouse brain atlas is a genome-wide, high-resolution atlas of gene expression throughout the adult mouse brain. The atlas provides genome-wide in situ hybridization (ISH) image data for approximately 20,000 genes in adult mice. Each data set is processed through an informatics analysis pipeline to obtain spatially mapped quantified expression information. See Lein, E. et al., “Genome-wide atlas of gene expression in the adult mouse brain,” Nature 445, 168-176 (2007).

FIG. 34 illustrates how spatial mapping of three cell types using the AdRoit method quantitatively depicts the content in each spot. FIG. 35 provides the ISH images of the Wfs1, Prox2, and Rarres2 cell types from the Allen mouse brain atlas. A comparison of between FIG. 34 and FIG. 35 shows that the ISH images of FIG. 35 match the corresponding cell locations in FIG. 34.

Although illustrated and described above with reference to certain specific embodiments and an example, the present disclosure is nevertheless not intended to be limited to the details shown. Rather, various modifications may be made in the details within the scope and range of equivalents of the claims and without departing from the spirit of the disclosure.

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

What is claimed is:

1. A method for deconvolving bulk or spatial RNA-sequencing data, the method comprising any one or more of the following steps:
a) obtaining input from sources comprising bulk or spatial RNA-seq data, single cell RNA-seq data, and cell type annotations, and selecting a subset of the most variably expressed genes from a normalized matrix of counts-based sequencing data;
b) estimating the mean and dispersion parameters of the expression per gene per cell type;
c) computing the cross cell type specificity of each gene;
d) estimating cross-sample gene variability from the bulk or spatial RNA-seq data or single cell samples depending on multi-sample availability;
e) estimating gene-wise scaling factors using both the bulk or spatial RNA-seq data and single cell data; and
f) building a weighted and regularized regression model using all of the known quantities and using the model to estimate cell type proportions in the bulk or spatial RNA-sequencing data;
thereby inferring the percentage of cell types in the bulk or spatial RNA-sequencing data.

2. The method according to claim 1, wherein the matrix of counts-based sequencing data comprises counts-based sequencing counts against each gene within a plurality of genes for a fixed number of cells.

3. The method according to claim 1, wherein the input is a single cell UMI count matrix with cell type annotations associated with each cell.

4. The method according to claim 1, wherein the bulk data to be deconvolved is transcripts per kilobase million (TPM) or read counts.

5. The method according to claim 1, wherein the spatial data to be deconvolved is a UMI count matrix.

6. The method according to claim 1, wherein genes that contain important information to differentiate cell types, excluding non-informative genes that potentially introduce noise, are selected as the subset of the most variably expressed genes from a normalized matrix of counts-based sequencing data.

7. The method according to claim 6, wherein the subset of genes is selected from the union of marker genes whose expression is enriched in each cell type in the single cell UMI count matrix.

8. The method according to claim 7, wherein a build-in tool takes as input the single cell UMI count matrix and cell type annotations.

9. The method according to claim 8, wherein for each cell type, the tool computes the fold change between the average UMI in that cell type and the average UMI in all other cell types, and ranks genes by descending fold changes.

10. The method according to claim 9, wherein about the top 200 genes from each cell type are selected.

11. The method according to claim 9, wherein selected marker genes presenting in no more than five cell types are selected.

12. The method according to claim 9, wherein selected marker genes presenting in a fixed number of cell types or a proportion of total number of cell types, whichever is smaller, are selected.

13. The method according to claim 9, wherein about 1,000 total unique genes are selected.

14. The method according to claim 6, wherein the subset of genes is selected from the union of highly variable genes that vary the most across all the cells in the single cell UMI count matrix.

15. The method according to claim 14, wherein variances for each gene after cell number balancing and VST normalization are computed.

16. The method according to claim 15, wherein genes with the highest variances are selected.

17. The method according to claim 14, wherein the cell types in the single cell UMI count matrix are balanced by finding the median size of all the cell clusters, wherein cells from each cluster are sampled to make them equal to this size.

18. The method according to claim 17, wherein the variance of each gene across the cells in the balanced single cell UMI matrix is computed.

19. The method according to claim 18, wherein variances on the normalized data are computed by variance stabilization transformed (VST).

20. The method according to claim 19, wherein genes with the top 2,000 large variances are selected.

21. The method according to claim 1, wherein the RNA-seq data is not normalized before estimating the mean.

22. The method according to claim 21, wherein the mean is modeled using the raw UMI counts.

23. The method according to claim 21, wherein negative binomial distributions are fit to single cells of each cell type.

24. The method according to claim 23, wherein estimations are performed for each selected gene in each cell type.

25. The method according to claim 1, wherein to compute the cell type specificity of a gene, the cell type in which the gene has: i) the highest expression, or ii) highest fold change compared to others, is identified, and the specificity of this gene is defined as the mean-to-variance ratio within the cell type.

26. The method according to claim 25, wherein the estimated mean and variance parameters from the negative binomial fitting is used to compute the cell type specificity weight for each gene in the set of selected genes.

27. The method according to claim 1, wherein the cross-sample gene variability is computed using the variance-to-mean ratio (VMR) computed across samples.

28. The method according to claim 27, wherein the cross-sample gene variability is computed from compound transcriptome data.

29. The method according to claim 27, wherein the compound data do not have multi-samples whereas the single cell data have multiple samples, and multiple compound samples are synthesized, each of which is an average of all cells belonging to one of the samples in the single cell reference.

30. The method according to claim 27, wherein if multi-samples are unavailable for both compound data and single cell data, the method comprises generating multiple synthetic samples for the single cell data by averaging the expression of subset of cells.

31. The method according to claim 1, wherein the gene-wise scaling factors are estimated using an adaptive learning strategy, and each gene is rescaled with its respective scaling factor.

32. The method according to claim 1, wherein each compound sample is independently estimated by the regression model.

33. A method for deconvolving bulk or spatial RNA-sequencing data, the method comprising any one or more of the following steps:
a) computing the cross cell type specificity of each gene within a subset of the most variably expressed genes selected from a normalized matrix of counts-based sequencing data obtained from sources comprising bulk or spatial RNA-seq data, single cell RNA-seq data, and cell type annotations;
b) estimating cross-sample gene variability from the bulk or spatial RNA-seq data or single cell samples depending on multi-sample availability;
c) estimating gene-wise scaling factors using both the bulk or spatial RNA-seq data and single cell data; and
d) building a weighted and regularized regression model using all of the known quantities and using the model to estimate cell type proportions in the bulk or spatial RNA-sequencing data;
thereby inferring the percentage of cell types in the bulk or spatial RNA-sequencing data.

34. A computer readable medium storing processor-executable instructions adapted to cause one or more computing devices to deconvolve bulk or spatial RNA-sequencing data by a method comprising any one or more of the following steps: i) obtaining input from sources comprising bulk or spatial RNA-seq data, single cell RNA-seq data, and cell type annotations, and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the expression per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities, and using the model to estimate cell type proportions in the bulk or spatial RNA-sequencing data; thereby inferring the percentage of cell types in the bulk or spatial RNA-sequencing data.

35. A computer readable medium storing processor-executable instructions adapted to cause one or more computing devices to deconvolve bulk or spatial RNA-sequencing data, by a method comprising any one or more of the following steps: i) computing the cross cell type specificity of genes within a subset of the most variably expressed genes selected from a normalized matrix of counts-based sequencing data obtained from sources comprising bulk or spatial RNA-seq data, single cell RNA-seq data, and cell type annotations; ii) estimating cross-sample gene variability from compound data or single cell samples, depending on multi-sample availability; iii) estimating gene-wise scaling factors using both compound data and single cell data; and iv) building a weighted and regularized regression model using all of the known quantities, and using the model to estimate cell type proportions in the bulk or spatial RNA-sequencing data; thereby inferring the percentage of cell types in the bulk or spatial RNA-sequencing data.

36. A system comprising:
one or more processors; and
a memory having processor executable instructions that, when executed by the one or more processors, cause the apparatus to deconvolve bulk or spatial RNA-sequencing data by a method comprising any one or more of the following steps: i) obtaining input from sources comprising bulk or spatial RNA-seq data, single cell RNA-seq data, and cell type annotations, and selecting a subset of the most variably expressed genes from a normalized matrix of counts-based sequencing data, wherein the matrix of counts-based sequencing data comprises counts-based sequencing counts against each gene within a plurality of genes for a fixed number of cells; ii) estimating the mean and dispersion parameters of the data per gene per cell type; iii) computing the cross cell type specificity of genes; iv) estimating cross-sample gene variability from compound data or single cell samples, depending on multi-sample availability; v) estimating gene-wise scaling factors using both compound data and single cell data; and vi) building a weighted and regularized regression model using all of the known quantities and using the model to estimate cell type proportions in the bulk or spatial RNA-sequencing data; thereby inferring the percentage of cell types in the bulk or spatial RNA-sequencing data.

37. A system comprising:
one or more processors; and
a memory having processor executable instructions that, when executed by the one or more processors, cause the apparatus to deconvolve bulk or spatial RNA-sequencing data, by a method comprising: i) computing the cross cell type specificity of genes within a subset of the most variably expressed genes selected from a normalized matrix of counts-based sequencing data obtained from sources comprising bulk or spatial RNA-seq data, single cell RNA-seq data, and cell type annotations; ii) estimating cross-sample gene variability from compound data or single cell samples, depending on multi-sample availability; iii) estimating gene-wise scaling factors using both compound data and single cell data; and iv) building a weighted and regularized regression model using all of the known quantities and using the model to estimate cell type proportions in the bulk or spatial RNA-sequencing data; thereby inferring the percentage of cell types in the bulk or spatial RNA-sequencing data.
